(19) United States
(12) Patent Application Publication
(10) Pub. No.: US 2025/0092373 A1
(43) Pub. Date: Mar. 20, 2025

(54) CRISPR-MEDIATED DELETION OF FLI1 IN NK CELLS

(71) Applicant: The Regents of the University of California, Oakland, CA (US)

(72) Inventors: Timothy E. O'Sullivan, Los Angeles, CA (US); Luke Riggan, Los Angeles, CA (US); Joey H. Li, Los Angeles, CA (US)

(73) Assignee: The Regents of the University of California, Oakland, CA (US)

(21) Appl. No.: 18/729,647

(22) PCT Filed: Jan. 25, 2023
(86) PCT No.: PCT/US23/61248

§ 371 Date: Jul. 17, 2024

Related U.S. Application Data
(60) Provisional application No. 63/302,756, filed on Jan. 25, 2022.

(51) Int. Cl. C12N 9/22; C12M 1/42; C12N 5/0783

(52) U.S. Cl. CPC C12N 9/22; C12M 35/02; C12N 5/0646; C12N 2310/20; C12N 2501/125; C12N 2501/145; C12N 2501/22; C12N 2501/2302; C12N 2501/2303; C12N 2501/2304; C12N 2501/2315; C12N 2510/00

(57) ABSTRACT

Natural killer (NK) cells are innate lymphocytes that possess traits of adaptive immunity, such as memory formation. However, the molecular mechanisms by which NK cells persist to form memory cells are not well understood. Using single cell RNA sequencing, we identified two distinct effector NK cell (NKeff) populations following mouse cytomegalovirus (MCMV) infection. Ly6C− memory precursor (MP) NK cells displayed enhanced survival during the contraction phase in a Bcl2-dependent manner, and differentiated into Ly6C+ memory NK cells. Our studies show that a NK cell-intrinsic checkpoint is controlled by the transcription factor Fli1 which limits MP NK formation by regulating early effector NK cell fitness during viral infection. Building upon this discovery, we have designed methods and materials for modulating the molecular mechanisms that regulate memory cell fate in NK cells, such as genetically modified NK cells having a deletion in the gene for the transcription factor Fli1.

### CROSS-REFERENCE TO RELATED APPLICATIONS

This application claims the benefit under 35 U.S.C. Section 119(e) of co-pending and commonly-assigned U.S. Provisional Patent Application Ser. No. 63/302,756, filed on Jan. 25, 2022, and entitled “CRISPR-MEDIATED DELETION OF FLI1 IN NK CELLS” which application is incorporated by reference herein.

### TECHNICAL FIELD

Embodiments of the disclosure concern at least the fields of immunology, cell biology, molecular biology, and medicine.

### BACKGROUND OF THE INVENTION

The ability of the immune system to remember previous pathogen encounters by executing a specific and robust secondary response upon re-exposure to pathogen-associated antigens is termed immunological memory. During infection, this memory response is largely performed by the selective clonal proliferation and long-term persistence of adaptive lymphocytes that express somatically recombined antigen receptors (e.g. T and B cells). Adaptive lymphocytes form antigen-specific memory cells that are able to epigenetically maintain activation-induced transcriptional changes following clearance of pathogens^{1, 2}. Coordination of stable epigenetic, transcriptional, and metabolic changes during T cell activation results in the cell-intrinsic capacity to form memory cells^{1, 3, 4}. The kinetics of the adaptive immune response to infection consist of three distinct phases: clonal expansion, contraction and memory formation. During the contraction phase 90-95% of all expanded effector cells are eliminated through cell-intrinsic apoptosis, leaving memory T and B cells that persist long-term in the host⁵. Previous studies have demonstrated that expanded CD8⁺ T cell populations consist of shorter-lived terminal effectors (TEs) that display decreased persistence during the contraction phase, and a smaller proportion of memory precursors (MPs) that contribute to the self-renewing memory CD8⁺ T cell pool⁵⁻⁸. However, whether MP-like cell states exist within the innate immune system is unknown.

Natural killer (NK) cells are circulating group 1 innate lymphocytes (ILCs) that play a critical role during herpesvirus infection in mice and humans⁹⁻¹¹. Although historically categorized as innate immune cells, circulating and tissue-resident group 1 ILCs can exhibit memory responses to mouse cytomegalovirus (MCMV)-associated glycoproteins through expression of germline encoded activating receptors¹²⁻¹⁴. Furthermore, NK cells exhibit clonal proliferation and persistence of a long-lived population of memory cells with enhanced protective capacity after secondary MCMV infection^{15, 16}. In C57BL/6 mice, a subset of naive Ly49H⁺ NK cells initiate this adaptive response after recognition of the MCMV-encoded glycoprotein m157^{14, 17, 18}. In both mice and humans, stochastically expressed germline-encoded activating and inhibitory receptors as well as developmental subsets generate naive NK cell diversity and heterogeneity during homeostasis^{12, 19, 20}. Furthermore, heterogeneity within the naive Ly49H⁺ NK cell pool can influence NK cell responses to MCMV. This involves preferential expansion of NK cells with a history of recombination-activating gene (RAG) expression, NK cells that lack expression of killer cell lectin-like receptor G1 (KLRG1) or the inhibitory receptor NKR-P1B²¹⁻²³, and NK cells with a longer history of NKp46 expression²⁴. Although subsets of naive NK cells have been found to preferentially form Ly49H⁺ effector NK (NK_{eff}) cells following MCMV infection, whether similar heterogeneity exists within expanded NK_{eff} cells remains unresolved. Furthermore, whether a subset of NK_{eff} cells preferentially survives to form memory cells, and the molecular mechanisms that regulate memory cell fate in NK cells remain poorly understood.

In view of the above, methods and materials for modulating the molecular mechanisms that regulate physiological processes such as memory cell fate in NK cells are needed.

### SUMMARY OF THE INVENTION

Natural killer (NK) cells are innate lymphocytes that possess traits of adaptive immunity, such as memory formation. However, the molecular mechanisms by which NK cells persist to form memory cells are not well understood. Using single cell RNA sequencing, we identified two distinct effector NK cell (NKeff) populations following mouse cytomegalovirus (MCMV) infection. Ly6C− memory precursor (MP) NK cells displayed enhanced survival during the contraction phase in a Bcl2-dependent manner, and differentiated into Ly6C⁺ memory NK cells. MP NK cells exhibited distinct transcriptional and epigenetic signatures compared to Ly6C+ NKeff cells, with a core epigenetic signature shared with MP CD8+ T cells enriched in ETS1 and Fli1 DNA-binding motifs. Fli1 was induced by STAT5 signaling ex vivo, and increased levels of the pro-apoptotic factor Bim in early effector NK cells following viral infection. These results provide evidence that a NK cell-intrinsic checkpoint controlled by the transcription factor Fli1 limits MP NK formation by regulating early effector NK cell fitness during viral infection. Building upon this discovery, we have designed methods and materials for modulating the molecular mechanisms that regulate memory cell fate in NK cells.

As discussed below, we have designed strategies for modulating molecular mechanisms that regulate memory cell fate in NK cells by perturbing (e.g. deleting) the gene for the transcription factor Fli1 in such cells. Typically such strategies involve CRISPR-Cas9 ribonucleoprotein (cRNP) genomic editing of the NK cells. Such methods can be used to modulate the physiology of early effector NK cells, for example so that levels of the pro-apoptotic factor Bim in these cells are decreased following viral infection. Related embodiments of the invention include methods of decreasing levels of the pro-apoptotic factor Bim in early effector NK cells following viral infection, the methods comprising deleting the FLI1 gene in the early effector NK cells such that levels of the pro-apoptotic factor Bim in the early effector NK cells are decreased following viral infection.

Illustrative working embodiments of the invention include methods of making a genetically modified NK cell, the methods comprising making a perturbation such as a deletion in the FLI1 gene of the NK cell so that the genetically modified NK cell is made. In embodiments of the invention, the method comprises deleting the FLI1 gene in the early effector NK cells such that levels of the pro-apoptotic factor Bim in the early effector NK cells are decreased following viral infection. Typically in these methods, the deletion in the FLI1 gene is made using a clustered regularly interspaced short palindromic repeats (CRISPR) process. For example, in some embodiments of the invention, these NK cell are made using a CRISPR process that comprises combining a CRISPR ribonucleoprotein complex with the NK cells, and then electroporating this combination under conditions comprising a voltage between 1700-2000 volts; and a pulse width of width of about 1×20-30 milliseconds such that the CRISPR ribonucleoprotein complex is electroporated into the NK cells. In certain embodiments of the invention, the NK cells are combined with one or more cytokines, for example following collection and prior to electroporation; wherein the one or more cytokines is selected from: IL-2, IL-3, IL-4, IL-15, the notch ligand DLL1, stem cell factor (SCF), FLT3 ligand (FLT3L), thrombopoietin (TPO), GM-CSF, and M-CSF.

Embodiments of the invention also include isolated NK cells having a deletion in the FLI1 gene such that levels of the pro-apoptotic factor Bim in the NK cells are decreased following expansion in vitro. In illustrative embodiments of the invention, the NK cell is a mature naive NK cell. Optionally, the NK cell comprises a further modification to genomic DNA comprising a further deletion of one or more genes in the NK cell and/or the addition of one or more genes in the NK cell.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF DRAWINGS

FIG. 1. Single cell RNA sequencing reveals two clusters of NK_{eff} cells following MCMV infection. Ly49H⁺ NK cells were transferred into naive Ly49H^{-/-} mice and infected with MCMV i.p. 16 hours later. TCRβ⁻CD3ϵ⁻ NK1.1⁺Ly49H⁺KLRG1⁺ NK cells were sorted on D7 and D14 PI. Cells were immediately processed using 10× Genomics Chromium droplet single cell RNA sequencing. (a) UMAP plot of 2,430 D7 PI NK_{eff} cells colored by cluster identities. Each dot represents an individual cell. (b) Gene Ontology (GO) enrichment analysis of upregulated genes in cluster 0 compared to cluster 1 from D7 PI NK_{eff} cells. Terms were considered statistically significantly enriched if −log₁₀(p_{adj})<0.05. (c) Violin plots showing the relative expression of six differentially expressed genes in the two clusters of D7 PI NK_{eff} cells. Each dot represents a cell. (d) UMAP plot of 4,399 cells combined from D7 and D14 PI NK_{eff} cells, colored by timepoint analyzed (top) and cluster identity based on differential gene expression (bottom). Each dot represents an individual cell. (e) UMAP plot showing memory precursor (MP) and terminal effector (TE) CD8⁺ T cell gene module scores (top) and violin plots showing the distribution of MP and TE gene module scores for each cluster (bottom).

FIG. 2. Ly6C⁻ NK_{eff} cells preferentially persist following MCMV infection. Splenic Ly49H⁺ NK cells were transferred into Ly49H^{-/-} mice i.v. and infected i.p with MCMV 16 hours after adoptive transfer. (a) Histograms of indicated cell surface marker expression from D7 PI Ly49H⁺ NK_{eff} cells (red) compared to isotype controls (black). (b) Experimental schematic. A mixture of purified WT splenic CD45.1 and CD45.2 Ly49H⁺ NK cells were transferred into Ly49H^{-/-} mice and infected i.p with MCMV 16 hours after adoptive transfer. On D7 PI, TCRβ⁻CD3ϵ⁻ NK1.1⁺Ly49H⁺KLRG1⁺CD45.1⁺Ly6C⁺ and TCRβ⁻CD3ϵ⁻ NK1.1⁺Ly49H⁺KLRG1⁺CD45.2⁺Ly6C⁻ NK_{eff} were sorted and then transferred into naive Ly49H^{-/-} hosts at a 1:1 ratio. Recipient spleens were harvested 12 days post transfer and analyzed by flow cytometry. (c) Representative flow cytometry plots and statistical analysis of co-adoptively transferred Ly6C⁺ (CD45.1) and Ly6C⁻ (CD45.2) Ly49H⁺ NK cell subsets before transfer (post sort) and harvested from recipient Ly49H/^{-/-} mice on D12 (post transfer). Change from post sort frequency (middle) and numbers (right) of recovered cells 12 days post transfer. (d) Experimental schematic. Indicated CD45.1 and CD45.2 NK_{eff} cell subsets were transferred into Ly49H^{-/-} hosts that were previously adoptively transferred with CD45.1×CD45.2 naive NK cells and infected with MCMV 7 days prior. (e) Quantification of the change in frequency of co-adoptively transferred Ly6C⁺ (CD45.1) and Ly6C⁻ (CD45.2) Ly49H⁺ NK cells subsets harvested from recipient mice on D12 (post transfer). Data are representative of 2-3 independent experiments with n=3 mice per group. Samples were compared using two-tailed Student's t test with Welch's correction, assuming unequal SD, and data points are presented as individual mice with the mean±SEM (***p<0.001, ****p<0.0001).

FIG. 3. Ly6C⁻ NK_{eff} cells differentiate into Ly6C⁺ memory NK cells. (a-c) Ly49H⁺ NK cells were adoptively transferred into Ly49H^{-/-} mice and infected with MCMV i.p. 16 hours later. (a) Representative flow plots and (b) frequency of adoptively transferred Ly49H⁺ Ly6C⁻ NK cells at indicated timepoints following MCMV infection. (c) Absolute numbers of Ly6C⁺ and Ly6C⁻ Ly49H⁺ NK cells in the blood at indicated timepoints post MCMV infection. (d) Representative flow plots showing Ly6C expression in indicated adoptively transferred NK_{eff} cell subsets from FIG. 2C on D12 post transfer. (e) Frequency of Ly6C⁻ to Ly6C⁺ conversion or vice versa at D12 post transfer. Data are representative of 2-3 independent experiments with a-c,e: n=3 mice per group. Samples were compared using two-tailed Student's t test with Welch's correction, assuming unequal SD, and data points are presented as individual mice with the mean±SEM (*p<0.05, **p<0.01, ****p<0.0001).

FIG. 4. MP NK cells are transcriptionally distinct and require Bcl2 for optimal survival during the contraction phase of the response to MCMV. (a-b) Splenic Ly49H⁺ NK cells were transferred into Ly49H^{-/-} mice i.v. and infected i.p with MCMV 16 hours after adoptive transfer. Splenic TCR□⁻CD3ϵ⁻ NK1.1⁺KLRG1⁺Ly49H⁺Ly6C⁺ and TCR□⁻CD3ϵ⁻NK1.1⁺KLRG1⁺Ly49H⁺Ly6C⁻ NK_{eff} were sorted from recipient mice on D7 PI. Sorted NK cells were immediately processed for mRNA extraction, library preparation and sequencing. (a) Heatmap showing differentially expressed genes between Ly6C⁺ and Ly6C⁻ D7 PI NK_{eff} cells (p_{adj}<0.05). (b) Normalized read counts of selected genes shown in (a). (c) MFI of Bcl2 and Bim in D7 PI Ly6C⁺ and Ly6C⁻ NK_{eff} cells. (d) Representative histogram and (e) MFI of intracellular Bcl2 expression in naive (D0) and D7 PI Ly49H⁺ NK_{eff} cells. (f) Quantification of the frequency of intracellular cytochrome c negative cells following Bcl2 inhibition with ABT-199 in naive and D7 PI Ly49H⁺ NK_{eff} cells ex vivo. (g,h) IL-15 pre-activated congenically distinct NK cells were electroporated in the presence of either Rosa26 NTC cRNP (CD45.1) or Bcl2 cRNP (CD45.2) before being transferred i.v. into Ly49H-deficient recipients at a 1:1 ratio. (g) BCL2 protein expression shown by histogram of Bcl2 cRNP or Rosa26 NTC RNP CRISPR edited NK cells 3 days post gene edit ex vivo. (h) Recipient mice were infected i.p with MCMV 16 hours after adoptive transfer. Quantification of adoptively transferred Ly49H⁺ NTC cRNP or Bcl2 cRNP-edited NK cells in the blood of recipient mice at various timepoints PI. Data are representative of 2-3 independent experiments with n=3-4 mice per group. Samples were compared using two-tailed Student's t test with Welch's correction, assuming unequal SD, and data points are presented as individual mice with the mean±SEM (*p<0.05, **p<0.01, ****p<0.0001).

FIG. 5. MP NK cells share a core epigenetic signature with MP CD8⁺ T cells. Splenic Ly49H⁺ NK cells were adoptively transferred into Ly49H^{-/-} mice i.v. and infected i.p with MCMV 16 hours after adoptive transfer. Splenic TCR□⁻CD3ϵ⁻ NK1.1⁺KLRG1⁺Ly49H⁺Ly6C⁺ and TCR□⁻CD3ϵ⁻NK1.1⁺KLRG1⁺Ly49H⁺Ly6C⁻ NK_{eff} were sorted from recipient mice on D7 PI. Sorted NK cells were immediately processed for ATAC library preparation and sequencing. (a) Heatmap showing differentially accessible peaks between Ly6C⁺ and Ly6C⁻ NK_{eff} cells. Peaks with p_{adj}<0.15 were plotted. (b) GO enrichment analysis on genes related to differentially accessible peaks in Ly6C⁺ and Ly6C⁻ NK_{eff} cells. Terms were considered statistically significantly enriched if-log₁₀(p_{adj})<0.05. (c) Heatmap showing common differentially accessible peaks in the comparisons of Ly6C⁻ to Ly6C⁺ NK_{eff} cells and MP to TE CD8⁺ T cells (p_{adj}<0.05). (d) Normalized read counts of peaks related to selected genes which have differential accessibility between indicated NK_{eff} cells (top) and T cells (bottom). (e) HOMER motif analysis displaying common transcription factor binding site motifs in differentially accessible peaks between D7 PI Ly6C⁻ NK_{eff} and MP CD8⁺ T cells and D7 PI Ly6C⁺ NK_{eff} and TE CD8⁺ T cells. Samples were compared using two-tailed Student's t test with Welch's correction, assuming unequal SD, and data points are presented as individual mice with the mean±SEM (*p<0.05, **p<0.01, ***p<0.001).

FIG. 6. Fli1 is induced by IL-15-mediated STAT5 signaling in mature NK cells. (a) Normalized read counts from bulk RNA-seq analysis of Ly49H⁺ NK cells at indicated time points PI. (b) Normalized read counts of Fli1 in splenic NK cells 3 hours after indicated cytokine stimulation ex vivo. (c) Western blot showing Fli1 and β-actin loading control protein levels in unstimulated, IL-2-stimulated, or IL-15-stimulated splenic NK cells. (d) Upper two rows, ATAC-seq peaks in the Fli1 locus; Lower two rows, STAT5 ChIP-seq peaks in the Fli1 locus in unstimulated or IL-2/IL-15-stimulated splenic NK cells. (e) Western blot showing Fli1 and β-actin loading control protein levels in IL-15-stimulated+DMSO, or IL-15 stimulated+STAT5 inhibitor (CAS 285986-31-4) treated splenic NK cells. Samples were compared using two-tailed Student's t test with Welch's correction, assuming unequal SD. (a,b) data points are presented as individual mice with the mean±SEM (*p<0.05, **p<0.01). (c,e) Data represent 2-3 independent experiments with: n=3 mice pooled per data point with the mean±SEM (*p<0.05, ** p<0.01).

FIG. 7. Fli1 restricts the formation of MP NK cells during viral infection. (a) Protein levels of Fli1 shown by western blot of Fli1 cRNP or Rosa26 NTC cRNP CRISPR edited NK cells 3 days after editing ex vivo. (b-i) IL-15 pre-activated congenically distinct NK cells were electroporated in the presence of either Rosa26NTC cRNP (CD45.1) or Fli1 cRNP (CD45.2) before being transferred i.v. into Ly49H-deficient recipients at a 1:1 ratio. Recipient mice were infected i.p with MCMV 16 hours after adoptive transfer. (b) Quantification of adoptively transferred Ly49H⁺ NTC cRNP or Fli1 cRNP-edited NK cells in the blood of recipient mice at various timepoints PI. (c) The percentage and (d) number of Ly49H⁺ NTC cRNP or Fli1 cRNP-edited NK cells are quantified in the spleen, lung, liver, and blood of D28 recipient mice. (e) The percentage of Ly6C⁻ or Ly6C⁺ NK cells is shown in Ly49H⁺ NTC cRNP or Fli1 cRNP-edited NK cells at various timepoints PI. (f,g) Fli1 cRNP or Rosa26 NTC cRNP CRISPR edited NK cells were labeled with CTV dye before being transferred i.v. into Ly49H-deficient recipients and splenic NK cells were isolated on D3 PI. (f) Representative histograms displaying CTV dilution of Fli1 cRNP and NTC cRNP Ly49H⁺ NK cells. (g) Quantification of division, proliferation and expansion index calculated using FlowJo V10 software. (h,i) Splenic NK cells were isolated Day 3 PI. Dot plots displaying quantification of (h) % Ki-67⁺ and (i) MFI of Bim, Bcl2 and Bcl2/Bim ratio in Fli1 cRNP and NTC cRNP Ly49H⁺ NK cells. (a) Data represent 2 independent experiments with: n=3 mice per group. (b-i) Data represent 2-3 independent experiments with: n=3-4 mice per group. Samples were compared using two-tailed Student's t test with Welch's correction, assuming unequal SD, and data are presented as individual mice with the mean±SEM (*p<0.05, **p<0.01, ***p<0.001, ****p<0.0001)

### DETAILED DESCRIPTION OF THE INVENTION

In the description of embodiments, reference may be made to the associated figures which form a part hereof, and in which is shown by way of illustration a specific embodiment in which the invention may be practiced. It is to be understood that other embodiments may be utilized, and structural changes may be made without departing from the scope of the present invention.

As discussed below, we have discovered strategies for modulating molecular mechanisms that regulate memory cell fate in NK cells by perturbing (e.g. deleting) the gene for the transcription factor Fli1 in such cells. Typically such strategies involve CRISPR-Cas9 ribonucleoprotein (cRNP) genomic editing of the NK cells. Surprisingly, such methods can be used to modulate the physiology of early effector NK cells, for example so that levels of the pro-apoptotic factor Bim in these cells are decreased following viral infection. Aspects and embodiments of this invention are described below and also found in Riggan et al., Nature Immunology volume 23, pages 556-567 (2022) (this specific publication is referred to herein as “Riggan et al.”).

The invention disclosed herein has a number of embodiments. Embodiments of the invention include, for example, an isolated NK cell having a perturbation (e.g. a deletion) in a FLI1 gene such that levels or functional activities of the pro-apoptotic factor Bim in the NK cell are decreased (e.g. as compared to a control NK cell not having a perturbation in a FLI1 gene). Briefly, as is known in the art, Friend leukemia integration 1 transcription factor (FLI1) is a transcription factor also known as transcription factor ERGB. Seem for example, NCBI Reference Sequence: NM_001271012.1. The FLI1 gene maps to 128,686,535-128,813,267 in GRCh38 coordinates. BIM, also referred to as BCL2, is a mammalian regulator of cell death. See, for example, NCBI Reference Sequence: NM_001204106 4836 bp mRNA linear PRI 22 Jul. 2018; ACCESSION: NM_001204106, VERSION NM_001204106.1. the BCL2L11/Bim gene is located on 2q13.

Embodiments of the invention include methods of making a genetically modified NK cell, the methods comprising making a perturbation in the FLI1 gene of an NK cell (e.g. a primary NK cell obtained from the peripheral blood leukocytes of an individual) so that the genetically modified NK cell is made. In illustrative working embodiments of the invention, the methods comprise deleting the FLI1 gene in the early effector NK cells such that levels of the pro-apoptotic factor Bim in the early effector NK cells are decreased (as compared to early effector NK cells not having a perturbation in the FLI1 gene). Typically in these methods, the perturbation in the FLI1 gene is made using a clustered regularly interspaced short palindromic repeats (CRISPR) process. In certain of these embodiments, the CRISPR process comprises: combining a CRISPR ribonucleoprotein complex with the NK cells; and then electroporating this combination under conditions comprising: a voltage between 1700-2000 volts; and a pulse width of width of about 1×20-30 milliseconds; such that the CRISPR ribonucleoprotein complex is electroporated into the NK cells. In certain embodiments of these methods, the NK cells are combined with one or more cytokines following collection and prior to electroporation; wherein the one or more cytokines is selected from: IL-2, IL-3, IL-4, IL-15, the notch ligand DLL1, stem cell factor (SCF), FLT3 ligand (FLT3L), thrombopoietin (TPO), GM-CSF, and M-CSF.

Certain embodiments of the invention include using such methods to modulate memory cell fate in NK cells, for example by modulating levels of the pro-apoptotic factor Bim in NK cells. Illustrative embodiments of the invention include methods of decreasing levels/activities of the pro-apoptotic factor Bim in NK cells such as the levels of Bim that are observed in early effector NK cells following viral infection, the methods comprising perturbing a FLI1 gene in the early effector NK cells such that levels/activities of the pro-apoptotic factor Bim in the early effector NK cells are observed to be decreased following viral infection as compared to early effector NK cells not having a perturbation in the FLI1 gene. In certain embodiments of these methods, perturbing the FLI1 gene comprises making a deletion in the FLI1 gene made using a clustered regularly interspaced short palindromic repeats (CRISPR) process comprising: combining a CRISPR ribonucleoprotein complex with the early effector NK cells; and then electroporating the combination of (a) under conditions comprising: a voltage between 1700-2000 volts; and a pulse width of width of about 1×20-30 milliseconds; such that the CRISPR ribonucleoprotein complex is electroporated into the early effector NK cells. In some embodiments of the invention, the early effector NK cell is a mature naive NK cell. Optionally in these methods, an early effector NK cell is disposed within a culture media comprising one or more cytokines, wherein the one or more cytokines is selected from: IL-2, IL-3, IL-4, IL-15, the notch ligand DLL1, stem cell factor (SCF), FLT3 ligand (FLT3L), thrombopoietin (TPO), GM-CSF, and M-CSF. In some embodiments of these methods, the early effector NK cell comprises a further modification to genomic DNA comprising a further deletion of one or more genes in the early effector NK cell and/or the addition of one or more genes in the early effector NK cell.

As discussed in detail below, embodiments of the invention include, for example, methods of electroporating a CRISPR ribonucleoprotein complex into mammalian NK cells. As used herein, the phrase “CRISPR ribonucleoprotein complex” refers to a ribonucleoprotein complex having CRISPR-associated endonuclease activity. Exemplary CRISPR ribonucleoprotein complexes include CRISPR/Cas9 CRISPR-associated endonuclease activity and CRISPR/Cpf1 CRISPR-associated endonuclease activity. CRISPR/Cas9 gene targeting requires a custom single-lead RNA (sgRNA) consisting of a targeted sequence (crRNA sequence) and a Cas9 nucleic acid recruitment sequence (tracrRNA). The crRNA region is a sequence of about 20 nucleotides, homologous to one of the regions of the gene you are interested in, that will guide the activity of the Cas9 nuclease. As used herein, the phrase “CRISPR-associated RNA” refers to an RNA component that, when combined with a CRISPR-associated protein, results in an CRISPR ribonucleoprotein complex. Exemplary CRISPR ribonucleoprotein complexes include ribonucleoprotein complexes having an CRISPR-associated protein, such as CRISPR/Cas9 protein or CRISPR/Cpf1 protein. An exemplary CRISPR-associated RNA includes a gRNA, including a crRNA and tracrRNA, for CRISPR/Cas9 protein that forms the CRISPR/Cas9 endonuclease system. Another exemplary CRISPR-associated RNA includes a crRNA for CRISPR/Cpf1 protein that forms the CRISPR/Cpf1 endonuclease system. Examples of these CRISPR ribonucleoprotein complexes, the CRISPR-associated RNA and protein components, and CRISPR-associated endonuclease systems are disclosed in the following references: Collingwood, M. A., Jacobi, A. M., Rettig, G. R., Schubert, M. S., and Behlke, M. A., “CRISPR-BASED COMPOSITIONS AND METHOD OF USE,” U.S. patent application Ser. No. 14/975,709, filed Dec. 18, 2015, published now as U.S. Patent Application Publication No. US2016/0177304A1 on Jun. 23, 2016 and issued as U.S. Pat. No. 9,840,702 on Dec. 12, 2017; and Behlke, M. A. et al. “CRISPR/CPF1 SYSTEMS AND METHODS,” U.S. patent application Ser. No. 15/821,736, filed Nov. 22, 2017, and U.S. Patent Application Publication No. 20190032131, the contents of which are hereby incorporated by reference herein in their entirety.

As is known in the art, primary cells (e.g. NK Cells) are those directly removed from an individual (e.g. a cancer patient), as compared to cell lines which are permanently established cell cultures. Typically, the methods of the invention comprise combining the CRISPR ribonucleoprotein complex with NK cells; and then electroporating this combination under conditions comprising: a voltage between 1700-2000 volts; and a pulse width of width of about 1×20-30 milliseconds; such that the CRISPR ribonucleoprotein complex is electroporated into the leukocytes. Typically in these methods, the CRISPR ribonucleoprotein complex comprises from 40 to 100 pmol Cas9 complexed with from 120 to 300 pmol sgRNA. In this context, a gRNA is comprised of a tracrRNA and crRNA. In particular, the crRNA and tracrRNA can be fused into a single chimeric nucleic acid (a single-guide RNA, or sgRNA) or they can be separate nucleic acids. In certain embodiments of the invention, these methods comprise not more than 1, 2 or 3 individual electroporations. Typically in these methods, the electroporation of the CRISPR ribonucleoprotein complex into the primary leukocytes results in modulation of expression of a gene in the NK cells targeted by the sgRNA. Electroporation methods, materials and devices that can be used with embodiments of the invention are disclosed, for example in US Patent Application Publication Nos.: 20200332276, 20200171303, 20200131500, 20200115668, 20200048600, 20200048599, 20190382792, 20190292510, 20190284579, 20190125165, 20190100721, 20190093125, 20180340186, 20180179485, 20180155688, 20180066222, 20180064073, 20170348525, 20170298390, 20170218355, 20160215297, and 20160129246, the contents of which are incorporated herein by reference.

As discussed in detail below, using single cell RNA sequencing (scRNAseq), we identified an MP-like transcriptional state of NK_{eff} cells following MCMV-driven expansion in vivo. We provide further evidence that Ly6C⁻ NK_{eff} cells preferentially survive the contraction phase to generate memory NK cells in a Bcl2-dependent manner. MP NK cells displayed distinct transcriptional and epigenetic signatures compared to Ly6C⁺ NK_{eff} cells, with an overlapping epigenetic signature shared with MP CD8⁺ T cells enriched in ETS1 and Fli1 transcription factor binding motifs. Fli1 regulated MP NK cell formation by promoting Bim levels in early effector NK cells to decrease the fitness of expanding NK cells during viral infection. These results provide evidence that memory NK cells, similar to memory T cells, are generated by a subset of epigenetically distinct MP cells that preferentially survive during the contraction phase of the response to viral infection and identify Fli1 as a critical regulator of MP NK cell formation. Aspects and embodiments of the invention are discussed in the following sections.

Time-Resolved scRNA-Seq Analysis Reveals an MP-Like NK Cell State

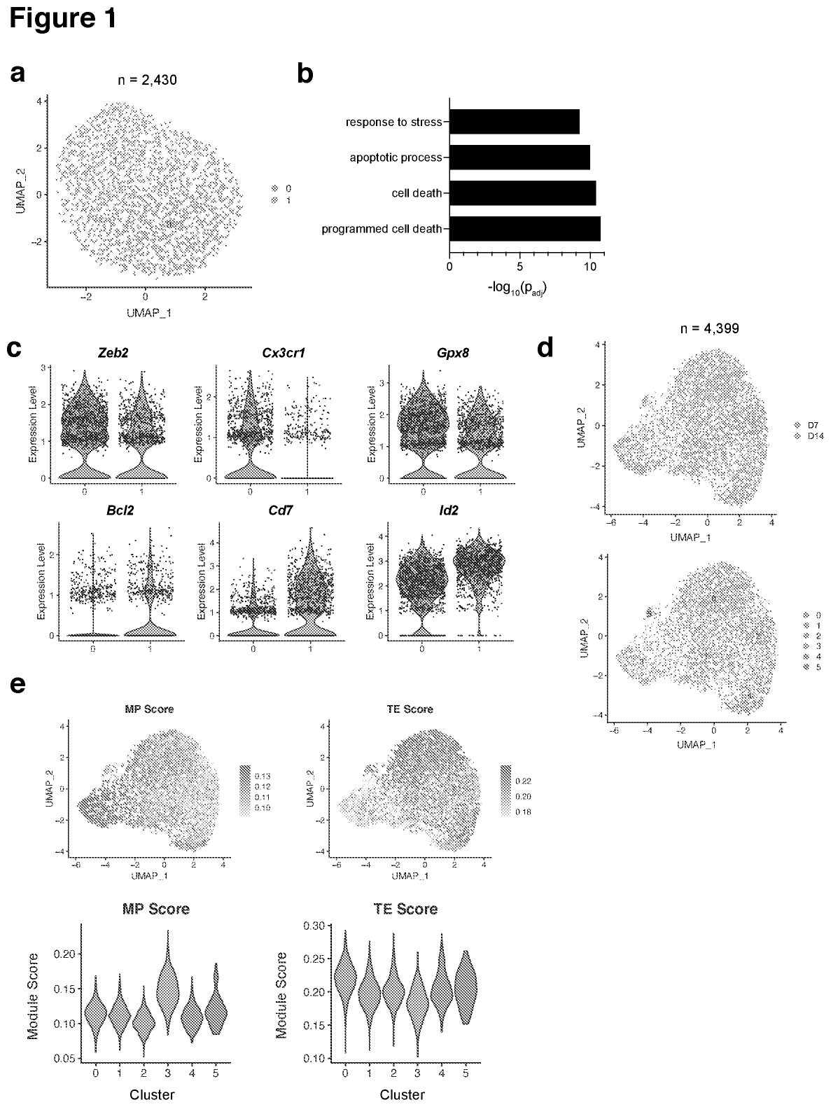

To determine whether transcriptional heterogeneity exists within NK_{eff} cells following viral infection, we adoptively transferred splenic Ly49H⁺ NK cells into recipient Klra8^{-/-} (hereafter referred to as Ly49H^{-/-}) mice and infected with MCMV. On day (D) 7 post-infection (PI), adoptively transferred NK_{eff} cells from the spleen were sorted and then profiled using 10× Genomics Chromium droplet scRNA-seq (Extended Data FIG. 1a in Riggan et al.). The resulting NK_{eff} single cell dataset included 2,430 cells that were clustered based on differential expression of marker genes and visualized using a uniform manifold approximation and projection (UMAP) plot (FIG. 1a, Supplementary Table 1 in Riggan et al.).). Clustering analysis revealed 2 distinct clusters of NK_{eff} cells defined by differential expression of transcription factors (Zeb2, Id2, Irf8, Runx3), regulators of cell survival (Gpx8, Shisa5, Bcl2), and cell surface proteins (Cx3cr1, Cd7, Ly6e) (FIG. 1c, Extended Data FIG. 1b in Riggan et al.). While previous studies have identified two subsets of naive mouse NK cells utilizing scRNA-seq¹⁹, these subsets did not account for the distinct clusters of NK_{eff} cells identified within our dataset, because all Ly49H⁺ NK cells analyzed displayed a mature phenotype (CD27⁻CD11b⁺) on D7 PI (Extended Data FIG. 1c in Riggan et al.).). Gene ontology enrichment analysis indicated that specific pathways were differentially regulated between the two NK_{eff} cell clusters. Notably, significant terms related to regulation of apoptosis were enriched in cluster 1 in comparison to cluster 0, with cluster 1 expressing more transcripts of the pro-survival gene Bcl2 than cluster 0 (FIG. 1b,c). These results provide evidence that transcriptional heterogeneity existed within NK_{eff} cells following viral infection, and that a specific effector cell state may preferentially persist during the contraction phase. Following MCMV-induced clonal proliferation, Ly49H⁺ NK cells undergo a contraction phase from D7 to D28 PI during which most NK_{eff} undergo Bim-mediated apoptosis²⁵. In order to address whether certain NK_{eff} cell states could persist during the contraction phase, adoptively transferred Ly49H⁺ NK cells were sorted on D14 PI and profiled using scRNA-seq. Combining this dataset with Ly49H⁺ NK cells analyzed from D7 PI resolved 4,399 cells that formed six distinct clusters which were visualized by UMAP dimensional reduction (FIG. 1d, Supplementary Table 2 in Riggan et al.). While the majority of cells comprising clusters 1,2 (Cx3cr1, Gpx8, Cma1) and 4 (Plac8, Gzmk, Thy1) were derived from D7 PI NK_{eff} cells, Cluster 0 (Cx3cr1, Sell, Cma1) and 5 (CD69, Ifng, Nfkbia) predominantly contained cells from D14 PI NK cells and resembled terminally-differentiated and activated NK cells respectively (FIG. 1d, Extended Data FIG. 1d in Riggan et al.). Notably, cluster 3 (Shisa5, Bcl2alb, Il2rb) contained a mix of cells from both D7 and D14 PI that were enriched in genes associated with response to stress and programmed cell death, suggesting that a distinct NK_{eff} cell state may persist during the contraction phase (FIG. 1d, Extended Data FIG. 1d,e in Riggan et al.).

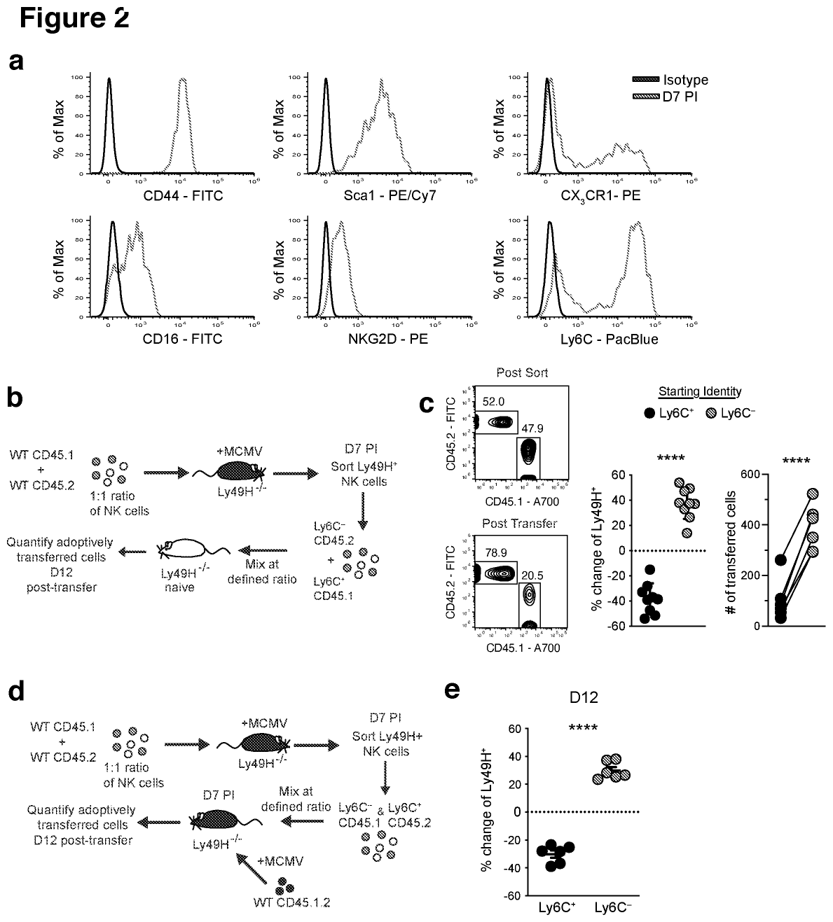

To test this hypothesis in silico, we utilized RNA velocity analysis²⁶ to determine the time-resolved transcriptional fates of D7 PI NK_{eff} cells (Extended FIG. 2a in Riggan et al.). Projection of the velocity field arrows onto the UMAP plot extrapolated future states of NK_{eff} cells showing 4 distinct manually averaged trajectories (Extended FIG. 2b in Riggan et al.). Both clusters 2 and 4 transitioned through cluster 1 to cluster 3 (trajectory 1), while a subset of cluster 3 cells did not show directionality to any other cell state (trajectory 2) suggesting that cluster 3 represented a distinct cell state of NK_{eff} (Extended FIG. 2a,b in Riggan et al.). RNA velocity analysis also suggested that cluster 0 represented an end stage of NK_{eff} differentiation as both cluster 2 (trajectory 3) and a subset of cells from cluster 3 (trajectory 4) showed strong directionality toward this cell state (Extended FIG. 2a,b in Riggan et al.). Monocle pseudotime analysis further corroborated in silico trajectories 1 and 4, showing that cluster 1 likely represented a transition state between cluster 4 and 3 on D7 PI, and that cluster 3 cells differentiated to cluster 0 on D14 PI (Extended FIG. 2c in Riggan et al.). Trajectory 1 was defined by increased expression of Bcl2 and Il2rb, while trajectory 4 was defined by increased expression of genes associated with NK cell terminal maturation such as Zeb2 and Irf8^{19, 27} (Extended FIG. 2d in Riggan et al.). Because cluster 3 represented a distinct NK_{eff} cell state that persisted during the contraction phase and could putatively differentiate to the majority of NK cells present on D14 PI, we then tested whether cluster 3 represented a MP-like NK cell state using MP and TE CD8⁺ T cell RNA-seq datasets described previously (GSE111902). Indeed, cluster 3 showed the highest enrichment for the MP CD8⁺ T cell gene expression module score, while cluster 0 scored the highest for the TE CD8⁺ T cell gene module score (FIG. 1e). Thus, these data suggest that D7 PI NK_{eff} cells contain a mixture of MP-like and other NK_{eff} cell states following viral infection.

Memory Ly6C⁺ NK Cells are Derived from Ly6C⁻ MP NK Cells

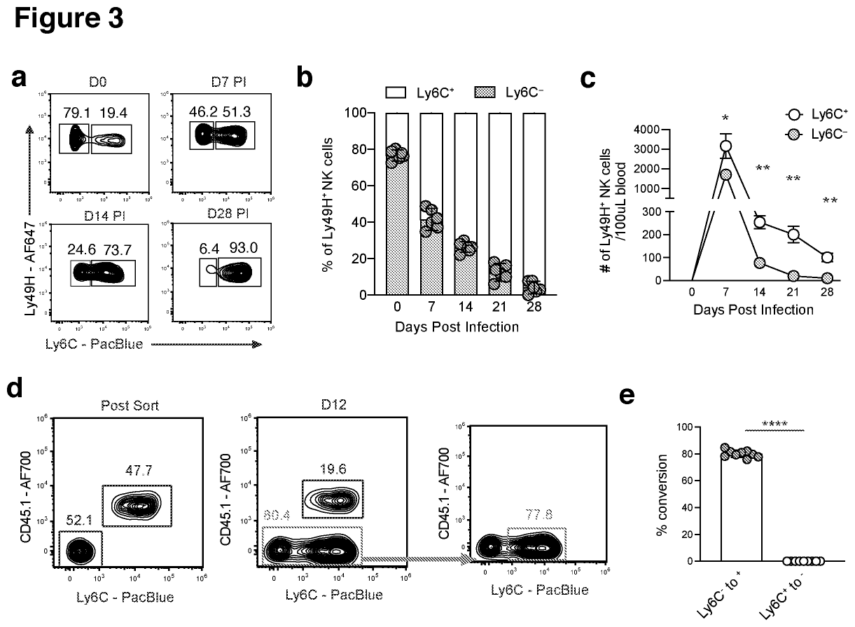

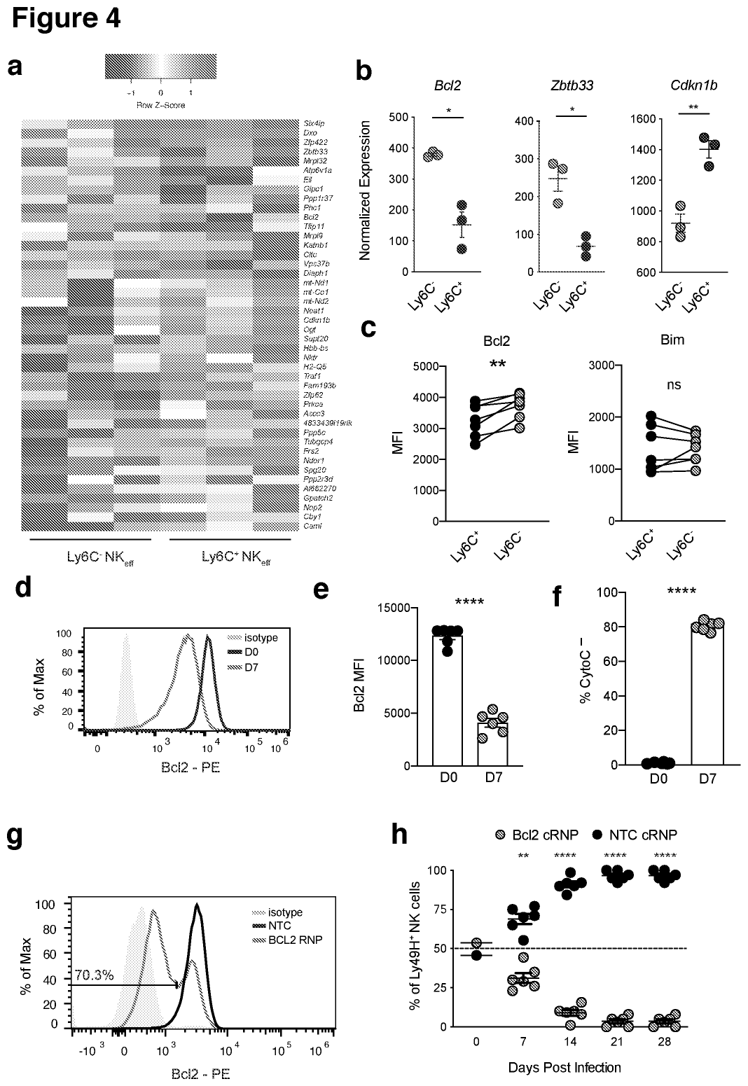

To determine whether a distinct MP NK cell subset could be identified in vivo, we analyzed cell surface expression of several genes predicted by our scRNA-seq dataset to be differentially expressed at D7 PI. While several cell surface proteins identified (CD44, Scal, NKG2D, CD16) did not display bimodal expression in effector NK cells, there were clear positive and negative populations of Ly6C and CX₃CR1 NK_{eff} cells on D7 PI (FIG. 2a). To test whether either of these markers could identify a subset of NK_{eff} cells with enhanced persistence during the contraction phase, we sorted congenically distinct Ly6C⁻ and Ly6C⁺ or CX₃CR1⁻ and CX₃CR1⁺Ly49H⁺ NK_{eff} cells at D7 PI and adoptively transferred equal mixtures of each into naive Ly49H^{-/-} mice (FIG. 2b). While we did not observe a change in the frequencies of CX₃CR1⁻ and CX₃CR1⁺ NK_{eff} cells 12 days post-transfer (Extended Data FIG. 3a in Riggan et al.), there was a significant increase in the frequency of recovered Ly6C⁻ compared to Ly6C⁺ NK cells on D12 following transfer. (FIG. 2c). These results were not due to differences in trafficking or proliferation of adoptively transferred Ly6C⁻ NK_{eff} cells, as the percentage of adoptively transferred Ly6C⁻ and Ly6C⁺ Ly49H⁺ NK cells remained similar in peripheral organs at various timepoints PI, and we did not observe differences in the frequencies of Ki-67⁺ Ly6C⁻ or Ly6C⁺ Ly49H⁺ NK cells on D7, D10, or D14 PI (Extended Data FIG. 3b-d in Riggan et al.). We also did not observe differences in single cell metabolic heterogeneity using SCENITH²⁸, or differential induction of mitophagy through analysis of TMRE and MitoGreen staining between Ly6C⁺ and Ly6C⁻ NK_{eff} at various timepoints during the contraction phase²⁹ (Extended Data FIG. 4a,b in Riggan et al.). Furthermore, D14 PI Ly6C⁺ and Ly6C⁻ NK_{eff} cells both displayed enhanced production of IFN-γ upon platebound antibody stimulation, and adoptively transferred D7 PI Ly6C⁺ and Ly6C⁻ NK_{eff} cells both rescued Ly49H^{-/-} mice from lethal MCMV challenge (Extended Data FIG. 4c,d in Riggan et al.). The observed enhanced survival phenotype was found to be intrinsic to Ly6C⁻ NK_{eff} cells, as co-adoptive transfer of congenically distinct Ly6C⁻ and Ly6C⁺ NK_{eff} cells led to a higher ratio of Ly6C⁻ to Ly6C⁺ NK cells 12 days post transfer into distinct Ly49H^{-/-} hosts that previously received adoptively transferred naive NK cells and were infected with MCMV (FIG. 2d,e). These results suggest that Ly6C⁻ NK_{eff} cells persist to a greater extent than Ly6C⁺ NK_{eff} cells during the contraction phase to preferentially form memory NK cells.

Previous studies have suggested >95% of MCMV-induced memory NK cells are Ly6C⁺ on D28 PI^{30, 31}. However, our adoptive transfer data suggested that Ly6C⁻ NK_{eff} give rise to the majority of memory NK cells present 12 days following transfer, suggesting that Ly6C⁺ NK_{eff} likely do not represent MP NK cells (FIG. 2c,e). Notably, RNA velocity analysis indicated that a subset of MP-like NK cells transitioned to a terminal cell state that represented the majority of memory NK cells on D14 PI (Extended Data FIG. 2a-d in Riggan et al.). These results suggested that the Ly6C⁻ NK_{eff} population was likely enriched in the MP-like NK cell state identified by our scRNA-seq analysis. In support of this hypothesis, the percentage and numbers of adoptively transferred Ly6C⁻ Ly49H⁺ NK cells declined over time in the blood (FIG. 3a-c) and peripheral organs by D28 PI (Extended Data FIG. 4e in Riggan et al.). Further examination of adoptively transferred Ly6C⁺ and Ly6C⁻ NK_{eff} cells demonstrated that while a majority of the surviving Ly6C⁺ memory NK cells were derived from D7 PI Ly6C⁻ NK_{eff} cells 12 days post transfer, ˜80% of D7 Ly6C⁻ NK_{eff} cells upregulated Ly6C while Ly6C⁺ NK_{eff} did not lose expression of Ly6C (FIG. 3d,e). These results suggest that D7 PI Ly6C⁻ NK_{eff} cells (referred to as MP NK cells hereafter) preferentially persist and differentiate into Ly6C⁺ memory NK cells during the contraction phase of the response to MCMV.

### Bcl2 is Required for the Survival of NK_{eff} Cells During Contraction

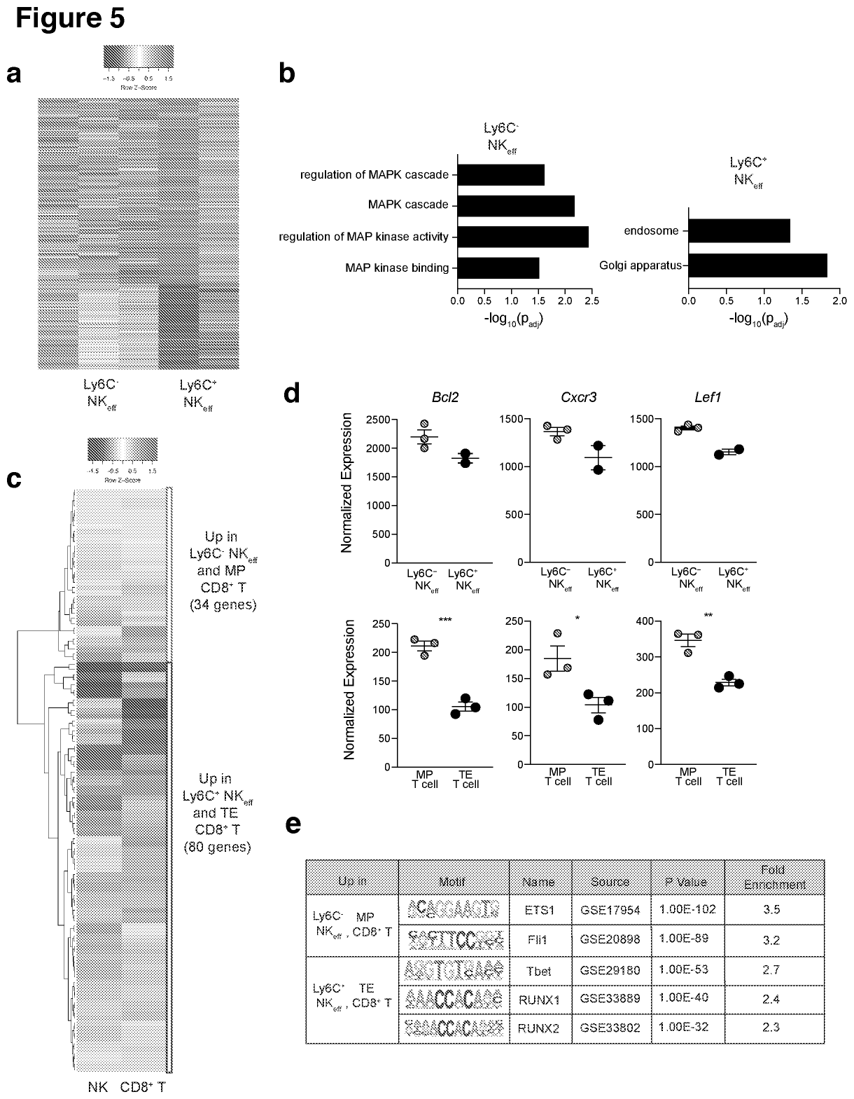

To determine the mechanisms of MP NK cell persistence following MCMV infection, we adoptively transferred CD45.1⁺Ly49H⁺ NK cells into Ly49H^{-/-} mice, infected with MCMV, sorted Ly6C⁺ and Ly6C⁻ Ly49H⁺ NK cells from the spleens of the Ly49H^{-/-} mice on D7 PI and performed RNA sequencing (RNA-seq). RNA-seq analysis identified 43 differentially expressed genes (DEGs) between Ly6C⁺ NK_{eff} and MP NK cells, with MP NK cells displaying increased expression of Bcl2 and the transcription factor Zbtb33 (FIG. 4a,b and Supplementary Table 3 in Riggan et al.). Conversely, Ly6C⁺ NK_{eff} cells displayed increased expression of the cell cycle inhibitor Cdkn1b (FIG. 4b). Analysis of common DEGs between MP vs TE CD8⁺ T cells and MP NK cells vs Ly6C⁺ NK_{eff} cells revealed higher expression levels of Bcl2 between MP CD8⁺ T cells and MP NK cells (Extended FIG. 5a in Riggan et al.). However, MP CD8⁺ T cells did not share an overlapping core transcriptional signature with MP NK cells. Instead, MP CD8⁺ T cells expressed high levels of Tcf7, Id3, P2rx7, and Espn, which have been associated with early memory T cell progenitor stemness programs³² (Extended FIG. 5b in Riggan et al.). In confirmation of our RNA-seq dataset, MP NK cells expressed higher levels of the pro-survival molecule Bcl2³³, whereas pro-apoptotic Bim levels were similar when compared to Ly6C⁺ NK_{eff} (FIG. 4c). Notably, bulk D7 PI NK_{eff} cells displayed lower levels of Bcl2 compared to naive NK cells (FIG. 4d,e), and were highly dependent on Bcl2 activity for survival as evidenced by increased release of intracellular cytochrome c following Bcl2 inhibition ex vivo (FIG. 4f). To determine whether Bcl2 was required for the persistence of Ly49H⁺ NK cells during the contraction phase, we generated high efficiency CRISPR ribonucleoprotein (cRNP)-mediated deletion of Bcl2 in primary mature NK cells³⁴ (FIG. 4g). Bcl2 cRNP-edited naive (CD45.2) Ly49H⁺ NK cells were co-adoptively transferred with control Rosa26-edited (CD45.1) Ly49H⁺ NK cells into Ly49H^{-/-}-mice and subsequently infected with MCMV. We observed a significant survival defect in Bcl2-deficient NK cells during the contraction phase (D7-D28 PI) (FIG. 4h). Together, these data suggest that higher levels of Bcl2 in MP NK cells may contribute to their enhanced persistence during the contraction phase of the response to MCMV infection.

### MP NK Cells Display a Distinct Epigenetic Signature

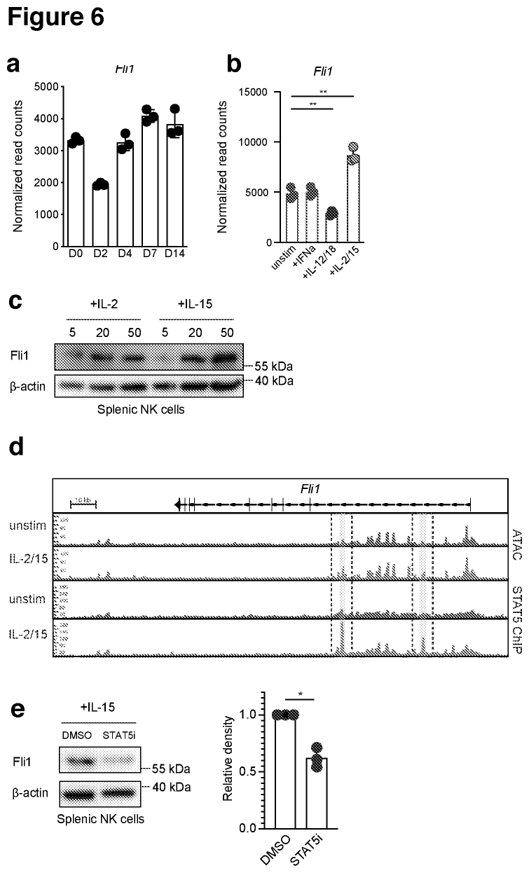

To determine the mechanisms of MP NK cell formation following MCMV infection, we adoptively transferred CD45.1⁺Ly49H⁺NK cells into Ly49H^{-/-} mice, infected with MCMV, sorted Ly6C⁺ and Ly6C⁻ Ly49H⁺ NK cells from the spleen of the Ly49H^{-/-} mice on D7 PI and performed ATAC sequencing (ATAC-seq). ATAC-seq analysis identified 811 significantly differentially accessible (DA) peaks between Ly6C⁺ NK_{eff} and MP NK cells (FIG. 5a, Supplementary Table 4 in Riggan et al.). Using gene ontology analysis, we found that MP NK cells displayed an increase in accessible peaks associated with genes implicated in MAPK signaling while Ly6C⁺ NK_{eff} cells showed increased accessibility in genes associated with the endosome and golgi (FIG. 5b). Next, we generated a core MP lymphocyte epigenetic signature consisting of 114 peaks that were significantly DA in MP CD8⁺ T and MP NK cells compared to TE CD8⁺ T cells and Ly6C⁺ NK_{eff} (FIG. 5c, Supplementary Table 5 in Riggan et al.). Peaks with greater accessibility in MP lymphocytes were associated with genes enriched in positive regulation of lymphocyte activation and differentiation such as the transcription factor Lefl, the chemokine receptor Cxcr3, and Bcl2 (FIG. 5d and Extended Data FIG. 6a-c in Riggan et al.). Together, these results suggest that MP NK cells display a common epigenetic signature shared with MP CD8⁺ T cells that is enriched in genes associated with T cell memory formation and effector function. To determine core transcriptional regulators of MP lymphocyte formation, we performed HOMER motif analysis of DA peaks shared between MP CD8⁺ T cells and MP NK cells. We found that MP DA peaks were enriched in ETS1 and Fli1 binding motifs compared to Ly6C⁺ NK_{eff} and CD8⁺ TE cell DA peaks that were enriched in Tbet, Runx1 and Runx2 binding motifs (FIG. 5e). These data indicated that MP NK cells display a distinct epigenetic signature compared to Ly6C⁺ NK_{eff}, and share a core epigenetic signature with MP CD8⁺ T cells.

Our ATAC-seq analysis suggested that Fli1 may be a critical transcriptional regulator of MP NK cells. Therefore, we examined whether mature NK cells express Fli1 during MCMV infection. RNA-seq analysis demonstrated that Fli1 was repressed early during viral infection, but subsequently increased during the expansion phase of the response on D4 PI (FIG. 6a). A recent study found that IL-2 and IL-15 levels remain elevated in MCMV-infected tissues until D4 PI³⁵, suggesting that these cytokines may induce Fli1 expression. Using the RNA-seq datasets from this study, we determined that splenic NK cells stimulated with IL-2 and IL-15 ex vivo induced Fli1 transcripts while IL-12 and IL-18 stimulated NK cells decreased Fli1 expression (FIG. 6b). To determine whether IL-2 or IL-15 induce Fli1 levels in mature NK cells, we examined Fli1 protein by immunoblot of purified splenic NK cells after 48 h culture with increasing doses of IL-2 or IL-15. While we observed that Fli1 was induced by both IL-2 and IL-15 in a dose-dependent manner in splenic NK cells, IL-15 induced more Fli1 protein than IL-2 at each dose (FIG. 6c). Further analysis of previously published ATAC-seq and STAT5 ChIP-seq data sets³⁵ revealed that IL-2 and IL-15 stimulation of splenic NK cells increased accessibility of peaks within the Fli1 locus that overlapped with enriched STAT5 ChIP-seq peaks, suggesting that STAT5 may directly induce Fli1 expression in NK cells in response to IL-2 or IL-15 stimulation ex vivo (FIG. 6d). To test this possibility, we stimulated splenic NK cells with IL-15 in the presence of a STAT5 inhibitor or vehicle control. Indeed, STAT5 inhibition resulted in a ˜50% reduction of Fli1 protein (FIG. 6e), suggesting that Fli1 protein levels are induced in mature NK cells by STAT5 signaling via IL-15 and/or IL-2 ex vivo.

### Fli1 Increases BIM Levels in Early NK_{eff} to Limit MP NK Cell Formation

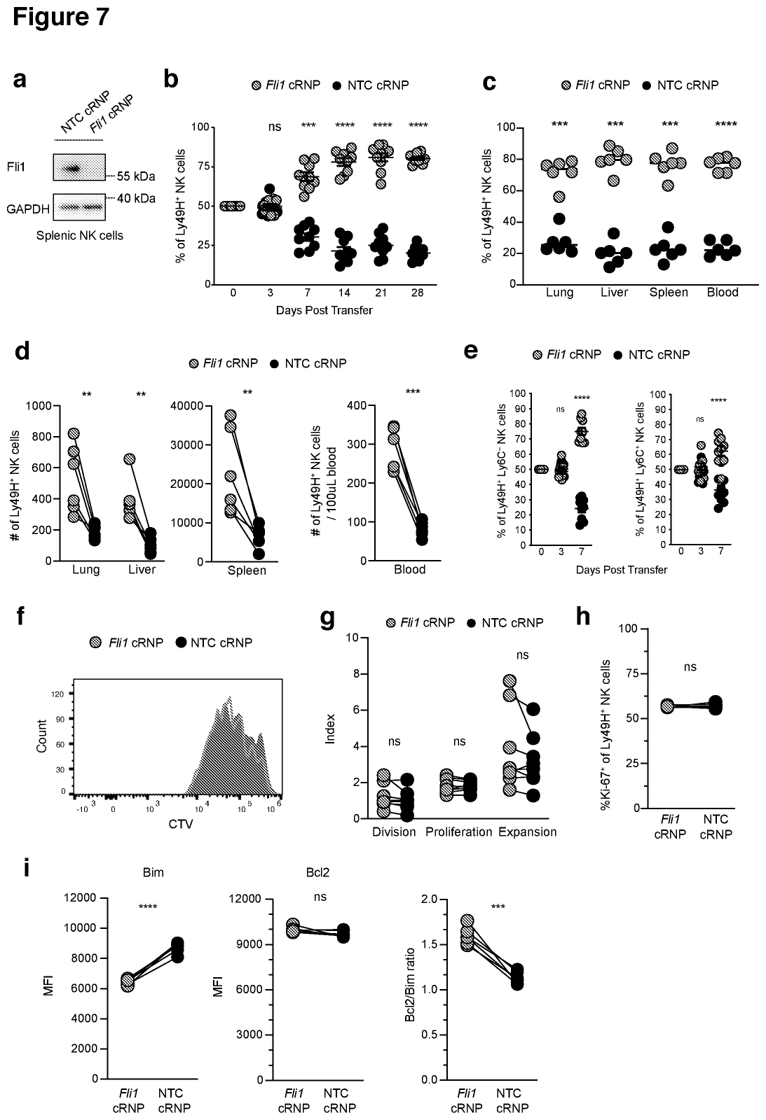

To determine the significance of Fli1 in regulating MP NK cells, we ablated Fli1 in mature NK cells using CRISPR cRNPs (FIG. 7a). We then adoptively transferred control or Fli1-edited Ly49H⁺ NK cells mixed at a 1:1 ratio into Ly49H^{-/-} mice and infected with MCMV 16 hours later. After D3 PI, Fli1-deficient NK cells represented a greater frequency of adoptively transferred Ly49H⁺ NK cells at all time points analyzed and persisted in greater numbers in the blood and peripheral organs on D28 PI (FIG. 7b-d). While the frequency of Fli1-deficient NK_{eff} cells increased compared to control edited cells on D7 PI, there was a greater impact of Fli1-deficiency on the frequency of MP NK cells compared to Ly6C⁺ NK_{eff}. These results suggested that Fli1 restricts the formation of MP NK cells in addition to limiting NK_{eff} formation (FIG. 7e). We hypothesized that the effects of Fli1 loss could be attributed to either increased proliferation or prolonged survival of adoptively transferred Ly49H⁺ cells. Analysis of adoptively transferred Fli1-deficient Ly49H⁺ NK cells on D3 PI did not reveal differences in CTV dilution, proliferation kinetics, or Ki-67 staining (FIG. 7f-h), suggesting that Fli1 does not control proliferation of Ly49H⁺ NK cells following viral infection. Instead, examination of Bim and Bcl2 protein by flow cytometry revealed that Fli1-deficient NK cells expressed significantly decreased levels of pro-apoptotic Bim versus control edited NK cells, while displaying similar levels of Bcl2 on D3 PI (FIG. 7i). This increased Bcl2/Bim ratio in Fli1-deficient NK cells coupled with their increased persistence in vivo suggested that Fli1 restricts the fitness of early effector NK cells through regulation of Bim levels during viral infection (Extended Data FIG. 7a,b in Riggan et al.).

### Discussion

We utilized single cell sequencing to identify a MP-like NK cell state following mouse cytomegalovirus (MCMV) infection. Ly6C⁻ NK_{eff} cells displayed enhanced survival during the contraction phase and were determined to be the main precursors of Ly6C⁺ memory NK cells. MP NK cells displayed distinct transcriptional and epigenetic signatures compared to Ly6C⁺ NK_{eff} cells, with increased protein expression of Bcl2. While Bcl2 was required for the survival of Ly49H⁺ NK cells during the contraction phase, STAT5 signaling likely induced Fli1 in early effector NK cells to increase Bim levels and restrict the formation of MP NK cells.

Time-resolved trajectory analysis of our scRNA-seq data suggested that a subset of D7 PI NK_{eff} cells transition to a cell state enriched in the MP CD8⁺ T cell transcriptional signature. Although bulk RNA-seq analysis demonstrated that MP CD8⁺ T cells and MP NK cells did not display overlapping gene signatures, they both display higher expression of Bcl2 transcript and protein, which is required for effector T cells to preferentially survive during the contraction phase of the anti-viral response^{36, 37}. These data suggest that Mcl1 expression, which is required for NK cell survival during homeostasis^{33, 38}, is not sufficient to inhibit apoptosis in NK_{eff} cells due to the decreased levels of Bcl2 observed on D7 PI. The discrepancy between our bulk RNA-seq and scRNA-seq data may be explained by the possibility that D7 PI Ly6C⁻ NK_{eff} cells contain a mixture of transitional NK_{eff} and MP NK cells rather than a homogenous MP cell state identified by scRNA-seq. Furthermore, because a small frequency of adoptively transferred Ly6C⁺ NK_{eff} persist during the contraction phase, it is possible that D7 PI Ly6C⁺ NK_{eff} contain a small fraction of transitional NK_{eff} that survive to generate memory NK cells on D14 PI. Irrespective of these points, our adoptive transfer data suggest that the majority of memory NK cells present on D19 PI are derived from D7 PI Ly6C⁻ NK_{eff} cells, making this a functional MP population.

RNA velocity analysis suggested that MP-like NK cells undergo continuous differentiation to a terminally differentiated state on D14 PI, which could explain the constant decay of memory NK cell numbers observed in all studies to date following the expansion phase in response to MCMV infection³⁹. Indeed, monocle trajectory analysis identified Zeb2 expression increasing towards the terminally differentiated memory NK cell state on D14 PI, and Zeb2 has been implicated in the terminal maturation of naive NK cells during homeostasis as well as the terminal differentiation of effector CD8⁺ T cells following viral infection^{27, 40, 41}. However, our results indicate that Zeb2 is required for the expansion of NK_{eff} cells, but not the terminal differentiation of MP-like NK cells (data not shown). Similarly, Id2 has been shown to maintain the terminal differentiation of effector CD8⁺ T cells through sustained repression of central memory-associated transcriptional programs in addition to regulating NK cell development and epigenetic regulation of effector functionality⁴²⁻⁴⁵. While we observed higher expression of Id2 in the D7 PI MP-like NK cell cluster from our scRNA-seq analysis, we did not observe an increase in MP NK cells following inducible deletion of Id2 during the contraction phase following MCMV infection (data not shown). Together, these findings suggest that there may be important epigenetic differences between effector NK and CD8⁺ T cells that dictate lineage specific terminal differentiation programs, although future studies will be necessary to support this hypothesis.

Our ATAC-seq dataset revealed that Ly6C⁺ NK_{eff} and TE CD8⁺ T cells share common enrichment in Runx1, Tbet, and Runx2 binding motifs, with Ly6C⁻ NK_{eff} and MP CD8⁺ T cells show enrichment for Fli1 and ETS1 binding motifs. While Tbet and Runx1 are important for NK cell survival during homeostasis and MCMV infection^{46,47}, the precise roles of these transcription factors during NK_{eff} terminal differentiation will need to be studied in further detail. In mouse and human NK cells, ETS-1 has been shown the be required for mature NK cell development and regulation of genes involved in apoptosis, and can be induced by IL-2/IL-15 signaling in human NK cells⁴⁸⁻⁵⁰. Similarly, we found that STAT5 signaling induced expression of Fli1 in mature mouse NK cells in response to IL-2 and IL-15 ex vivo. Interestingly, CRISPR-mediated deletion of Fli1 in mature naive NK cells leads to decreased Bim levels and a greater persistence of early effector NK cells following MCMV infection, with a larger proportion of MP NK cells persisting through the contraction phase as a result. This finding suggests that Fli1 acts as a repressor of MP NK cell formation, likely to limit bystander immunopathology of expanded Ly49H⁺ NK cells following clonal proliferation. Similarly, Fli1-deficient CD8⁺ T cells accumulate more effector cells following LCMV infection⁵¹, implicating Fli1 as a critical intrinsic checkpoint regulator of effector lymphocyte formation. While future studies will be needed to determine the epigenetic and/or transcriptional mechanisms by which Fli1 represses early effector lymphocyte fitness, these results identify an important regulator of effector lymphocyte formation. Thus, understanding the transcriptional and epigenetic pathways that induce MP states in NK cells could inform strategies aimed at enhancing adaptive NK cell adoptive immunotherapies.

### Method Details

### Mice

Mice were bred at UCLA in accordance with the guidelines of the Institutional Animal Care and Use Committee (IACUC). The following mouse strains were used this study: C57BL/6 (CD45.2) (Jackson Labs, #000664), B6.SJL (CD45.1) (Jackson Labs, #002114), Klra8^{-/-} (Ly49H-deficient). Experiments were conducted using 6-8 week old age- and gender-matched mice in accordance with approved institutional protocols.

### MCMV Infection

MCMV (Smith) was serially passaged through BALB/c hosts three times, and then salivary gland viral stocks were prepared with a dounce homogenizer for dissociating the salivary glands of infected mice 3 weeks after infection. Experimental mice in studies were infected with MCMV by i.p. injection of 7.5×10³ plaque-forming units (PFU) in 0.5 mL of PBS. In other experiments, Ly49H^{-/-} mice were intravenously injected with 25,000 sort purified D7 PI Ly49H⁺ NK cells or PBS as control. 7 days later, recipient mice were infected with MCMV by i.p. injection of 7.5×10³ plaque-forming units (PFU) in 0.5 mL of PBS. Mice were monitored and weighed daily and sacrificed when body weight decreased 10% from initial weight.

### Isolation and Enrichment of Mouse NK Cells

Mouse spleens, livers, lungs, lymph nodes, and blood were harvested and prepared into single cell suspensions as described previously¹¹. Splenic single cell suspensions were lysed in red blood cell lysis buffer and resuspended in EasySep™ buffer (Stemcell). To avoid depleting Ly6C⁺ NK cells we developed a custom antibody cocktail as follows: splenocytes were labeled with 10 μg per spleen of biotin conjugated antibodies against CD3ϵ (17A2), CD19 (6D5), CD8 (53-6.7), CD88 (20/70), Ly6G (1A8), SiglecF (S17007L), TCRβ (H57-597), CD20 (SA275A11), CD172a (P84) and magnetically depleted from total splenocyte suspensions with the use of anti-biotin coupled magnetic beads (Biolegend).

### Cell Sorting and Adoptive Transfer Experiments

Isolated splenic NK cells were sorted using Aria-H Cytometer. NK cells were sorted to >95% purity. Approximately 2×10⁵ enriched NK cells were injected intravenously into mice. In adoptive co-transfer experiments, equal numbers of Ly49H⁺ NK cells from each population (CD45.1⁺ and CD45.2⁺) were injected into recipients 16 hours prior to MCMV infection. In other experiments, TCRβ⁻CD3ϵ⁻ NK1.1⁺Ly49H⁺KLRG1⁺CD45.1⁺CX₃CR1⁺ and TCRβ⁻CD3ϵ⁻NK1.1⁺Ly49H⁺KLRG1⁺CD45.2⁺CX₃CR1⁻ or TCRβ⁻CD3ϵ⁻NK1.1⁺Ly49H⁺KLRG1⁺Ly6C⁺ and TCRβ⁻CD3ϵ⁻NK1.1⁺Ly49H⁺KLRG1⁺Ly6C⁻ D7 PI NK_{eff} were sorted and then transferred at a 1:1 ratio into naive or D7 PI Ly49H^{-/-} hosts that had received CD45.1×CD45.2 Ly49H⁺ NK cells 7 days prior to MCMV infection. Adoptively transferred cells were recovered by harvesting recipient mouse splenic NK cells, preforming magnetic enrichment, and analysis by flow cytometry at various time points post-transfer.

### Guide RNA Design

Synthetic gRNAs were purchased from SYNTHEGO. gRNA sequences were derived from a mouse whole genome CRISPR library described previously⁵². 10 gRNA sequences from this dataset were ranked according to predicted indel percentage and low off-target score using the inDelphi machine-learning algorithm for each gene target⁵³. The top 3-7 guides were validated for high indel percentages by Sanger sequencing and protein knockdown by western blot before utilization in experiments.

Electroporation and cRNP Complex Formation

gRNAs (Synthego) were diluted to 100 μM (100 pMol/μL) in 1× TE buffer (Sythego). 1.2 μL (120 pMol) of gRNA, 0.9 μL of 100 μM Alt-R® Cas9 Electroporation Enhancer (IDT) and 3.9 μL water were added to a 1.5 mL tube per sample for a total of 6 μL. 1 μL of recombinant Cas9 (20 pMol) (Synthego) was added to 5 μL water in a separate 1.5 mL tube. 6 μL of diluted Cas9 was added to 6 μL of gRNA-enhancer mixture for a total of 12 μL cRNP complex at a 1:3 molar ratio. The cRNP complex was allowed to incubate for at least 10 minutes at room temperature (RT) and electroporated using the Neon Transfection system (Thermo-Fisher) as described previously^{34, 54}. Cells were then incubated at 37° C. for either 10 minutes before adoptive transfer or 90 minutes before centrifugation and resuspension in complete media supplemented with 50 ng rmIL-15 for culturing in vitro. Cells were cultured in vitro for 3 days following electroporation prior to reading out gene editing efficiency by flow cytometry or sanger sequencing.

### Ex Vivo Stimulation of Lymphocytes

For plate-bound antibody stimulation experiments, ˜5×10⁵ isolated NK cells were stimulated with 4 mg/mL precoated antibody against NK1.1 (PK136) for 4 hours in complete media containing Brefeldin A (1:1000; BioLegend) and Monensin (2 uM; BioLegend). Cells were cultured in media alone as a negative control. In cytokine stimulation experiments, isolated NK cells were incubated with various concentrations of mouse IL-15 or IL-2, in the presence or absence of 100 μM CAS 285986-31-4 STAT5 inhibitor (Millipore Sigma).

Adoptive Transfer cRNP Experiments

Adoptive NK cell co-transfer studies were performed by injecting a total of 1×10⁶ NK cells; Rosa26 cRNP-edited WT, and gene x cRNP-edited WT NK cells purified from spleens of congenically distinct WT mice (CD45.1, CD45.1.2 or CD45.2) into Ly49H/^{-/-} mice 16 hours prior to MCMV infection.

### Proliferation Assays

CellTrace™ Violet (CTV) stock solution was prepared per the manufacturers' instructions (Thermo) and diluted at 1:1000 in 37 C. PBS. Isolated NK cells were incubated in 0.5 mL of diluted CTVsolution for 10 minutes at 37 C. The solution was quenched with 10× the volume of CR-10 media. Cells were then washed and injected i.v. Division, proliferation and expansion indexes were quantified using FlowJo V10 software using the following calculations. Division Index: Total Number of Divisions/The number of cells at start of culture. Proliferation Index: Total Number of Divisions/Cells that went into division. Division Index: Total Number of Divisions/The number of cells at start of culture.

### Flow Cytometry

Cells were analyzed for cell-surface markers using fluorophore-conjugated antibodies (BioLegend, eBioscience). Cell surface staining was performed in 1× PBS and intracellular staining was performed by fixing and permeabilizing using the eBioscience Foxp3/Transcription Factor kit for intranuclear proteins or BD Cytofix/Cytoperm kits for cytokines. Flow cytometry was performed using the Attune N×T Acoustic Focusing cytometer and data were analyzed with FlowJo software (BD). Cell surface and intracellular staining was performed using the following fluorophore-conjugated antibodies: CD45.1 (A20), CD45.2 (104), NK1.1 (PK136), KLRG1 (2F1), TCRβ (H57-597), CD3ϵ (17A2), Ly49H (3D10), IFN-γ (XMG1.2), Ly6C (HK1.4), CD44 (IM7), CD16 (93), Sca-1 (E13-161.7), CX3CR1 (SA011F11), NKG2D (CX5), BCL2 (BCL/10C4), CD11b (M1/70), CD27 (LG.3A10), Bim (C34C5), Ki-67 (16A8), Cytochrome-C (6H2.B4). For mitochondrial dyes, NK cells were enriched from spleens as described above, stained with cell-surface antibodies, and then incubated with various dyes in Hank's balanced salt solution plus Mg and Ca as follows: 100 nM Mitotracker Green (Life Technologies) for 30 min at 37°° C. to measure mitochondrial mass or 100 nM TMRE (Thermofisher) for 30 min at 37° C. to measure mitochondrial membrane potential. BH3 profiling was performed as previously described⁵⁵. Briefly, purified NK cells were resuspended in MEB buffer (150 mM Mannitol 10 mM HEPES-KOH, 50 mM KCl, 0.02 mM EGTA, 0.02 mM EDTA, 0.1% BSA, 5 mM Succinate). 50 μl of cell suspension (1×10⁵ cells/well) were plated in wells holding 50 μL MEB buffer containing 0.002% digitonin and BCL2 inhibitor ABT-199. Plates were then incubated at 25° C. for 50 min. Cells were then fixed with 4% paraformaldehyde for 10 min, followed by neutralization with N2 buffer (1.7M Tris, 1.25M Glycine pH 9.1) for 5 min. Samples were stained for 1 hour with 20 μL of staining solution (10% BSA, 2% Tween 20 in PBS) containing anti-cytochrome c (BioLegend). Immediately afterwards, cytochrome c release was quantified using Attune Flow Cytometer.

### Single Cell Metabolism Assay (SCENITH)

To profile single cell metabolic responses, 95 uL of purified NK cells in complete media were plated at 0.1−0.5×10⁶ cells/mL in v-bottom 96-well plates. Experimental triplicates were performed in all conditions. Wells were then treated with Control (DMSO), 2-Deoxy-D-Glucose (DG) final concentration 100 mM, Oligomycin (O) final concentration 1 mM, or a sequential combination of DG and O at the final concentrations mentioned. As negative control, the translation initiation inhibitor Harringtonine was added (Harringtonine, 2 mg/mL). Puromycin (final concentration 10 mg/mL) is added immediately after the metabolic inhibitor treatment. After puromycin treatment, cells were washed in cold PBS and stained for surface markers. Intracellular staining of puromycin was achieved using a custom anti-puromycin antibody²⁷ was performed by incubating cells during 1 h at 4° C. diluted in permeabilization buffer.

### PCR and Sanger Sequencing

DNA from NK cells was isolated using DNeasy Blood and Tissue kits (Qiagen). DNA concentration was measured using the NanoDrop OneC Microvolume UV-Vis Spectrophotometer (Thermo Scientific) and then diluted to 50 ng/μl in water before PCR amplification of cRNP-targeted genomic regions of approximately 500-1000 base pairs. PCR samples from WT and cRNP-edited cells were submitted for Sanger sequencing (GENEWIZ) and then indel percentage was calculated using ICE analysis (SYNTHEGO).

### 10× Library Preparation, Sequencing, and Alignment

Single cell RNA-sequencing libraries were generated with the Chromium Single Cell 3′ v2 (Day 7) and v3 (Day 14) assay (10× Genomics). Libraries were sequenced using the HighSeq 4000 platform (Illumina) to a depth of approximately 300 million reads per library with 2×50 read length. Raw reads were aligned to mouse genome (mm10) and cells were called using cellranger count (v3.0.2).

scRNA-Seq Cell Clustering

The R package Seurat (v3.1.)⁵⁶ was used to cluster the cells. Cells with less than 100 genes detected or more than 10% mitochondrial gene expression were first filtered out as low-quality cells. The gene counts for each cell were divided by the total gene counts for the cell and multiplied by a scale factor 10,000, then natural-log transformation was applied to the counts. The FindVariableFeatures function was used to select variable genes with default parameters. The ScaleData function was used to scale and center the counts in the dataset. Principal component analysis (PCA) was performed on the variable genes, and 20 principal components were used for cell clustering (resolution=0.5) and uniform manifold approximation and projection (UMAP) dimensional reduction. The cluster markers were found using the FindAllMarkers function. Module scores were calculated using the AddModuleScore function with default parameters. In the comparison between D7 and D14 PI NK cells, the FindIntegration Anchors and IntegrateData functions were used to find anchors and integrate the D7 and D14 PI datasets, and the other steps were the same as described above.

### RNA Velocity Analysis

To estimate the RNA velocities of single cells, velocyto²⁶ was used to distinguish unspliced and spliced mRNAs in each sample. The python package scVelo⁵⁷ was then used to recover the directed dynamic information by leveraging RNA splicing information. Specifically, the data was first normalized using the filter_and_normalize function. The first- and second-order moments were computed for velocity estimation using the moments function. The velocity vectors were obtained using the velocity function. The velocities were projected into a lower-dimensional embedding using the velocity_graph function. Finally, the velocities were visualized in the UMAP embedding using the velocity_embedding_stream function. All scVelo functions were used with default parameters.

### Pseudo-Time Trajectory Construction

Pseudo-time trajectories were constructed using the R package Monocle⁵⁸ (version 2.10.1). The raw counts for cells in the intended cell types were extracted and normalized by the estimateSizeFactors and estimateDispersions functions with the default parameters. Genes with average expression larger than 0.5 and detected in more than 10 cells were retained for further analysis. Variable genes were determined by the differentialGeneTest function with a model against the cell type identities. The top 2,000 variable genes with the lowest adjusted p value were used to order the cells. The orders were determined by the orderCells function, and the trajectory was constructed by the reduceDimension function with default parameters.

### Bulk RNA Sequencing

RNA was isolated from the cells using RNeasy Mini kit (Qiagen) and used to generate RNA-seq libraries followed by sequencing using Illumina HighSeq 4000 platform (single end, 50 bp). The reads were mapped with STAR⁵⁹ (version 2.5.3.a) to the mouse genome (mm10). The counts for each gene were obtained by using—quantMode GeneCounts commands in STAR, and the other parameters during alignment were set to default. Differential expression analyses were carried out using DESeq2⁶⁰ (version 1.24.0) with default parameters. Genes with adjusted p value <0.05 were considered significantly differentially expressed. Sequencing depth normalized counts were used to plot the expression values for individual genes. The T cell bulk RNA-seq datasets were downloaded from GEO (GSE111902)⁶¹, and the same DESeq2 procedure was applied. Genes with the absolute log2 fold change >0.5 and adjusted p value <0.05 in both NK cell and T cell datasets were plotted in FIG. 5C. Cytokine stimulated bulk NK RNA-seq datasets were downloaded from GEO (GSE140044)³⁵.

### ATAC-Seq Analysis

For ATAC-Seq, 50,000 cells per sample were lysed to collect nuclei and treated with Tn5 transposase (Illumina) for 30 minutes at 37° C. with gentle agitation. The DNA was isolated with DNA Clean & Concentrator Kit (Zymo) and PCR amplified and barcoded with NEBNext High-Fidelity PCR Mix (New England Biolabs) and unique dual indexes (Illumina). The ATAC-Seq library amplification was confirmed by real-time PCR, and additional barcoding PCR cycles were added as necessary while avoiding overamplification. Amplified ATAC-Seq libraries were purified with DNA Clean & Concentrator Kit (Zymo). The purified libraries were quantified with Kapa Library Quant Kit (KAPA Biosystems) and quality assessed on 4200 TapeStation System (Agilent). The libraries were pooled based on molar concentrations and sequenced on an Illumina HighSeq 4000 platform (paired end, 100 bp).

ATAC-seq fastq files were trimmed to remove low-quality reads and adapters using Cutadapt⁶² (version 2.3). The reads were aligned to the reference mouse genome (mm10) with bowtie2⁶³ (version 2.2.9). Peak calling was performed with MACS2⁶⁴ (version 2.1.1). The peaks from all samples were merged into a single bed file, peaks from different samples that were closer than 10 bp were merged into a single peak. HTseq⁶⁵ (version 0.9.1) was used to count the number of reads that overlap each peak per sample. The peak counts were analyzed with DESeq2⁶⁰ (version 1.24.0) to identify differentially accessible genomic regions. Peaks with adjusted p value <0.15 were considered significantly differentially accessible. The peak counts were visualized with IGV, version 2.5.0. The differentially accessible peaks were analyzed using the findMotifsGenome.pl function from homer⁶⁶ (version 4.9.1) to identify enriched cis-regulatory motifs of transcription factors. The T cell ATAC-seq datasets were downloaded from GEO (GSE111902), the same pipeline described above were used to analyze the datasets. Cytokine stimulated NK cell ATAC-seq datasets were downloaded from GEO (GSE140044) and visualized using the same pipeline described above. Peaks in the T cell ATAC-seq datasets with adjusted p value <0.05 were considered significantly differentially accessible. Significant peaks with the same change directions in the NK and T cell datasets were plotted in FIG. 6C.

### ChIP-Seq Analysis

STAT5 ChIP-seq datasets derived from IL-15/IL-2 stimulated splenic NK cells were downloaded from GEO (GSE140044)³⁵ and visualized using IGV, version 2.5.0.

### Western Blot

Protein was extracted from enriched primary splenic NK cells using Pierce RIPA buffer (Thermo-Fisher) with Halt protease inhibitor cocktail (Thermo-Fisher) and protein concentration was quantified using the Pierce BCA Protein Assay kit (Thermo-Fisher). Samples were electrophoresed on NuPage Novex 4-12% Bis-Tris Protein Gels, transferred to PVDF membranes, and blocked for one hour at room temperature with 5% w/v nonfat milk in 1× TBS and 0.1% Tween-20. Immunoblots were performed using rabbit anti-Fli1 (Abcam ab133485), rabbit anti-β-actin (Cell Signaling CST4970), and rabbit anti-GAPDH (Millipore Sigma G9545). Proteins were detected using the SuperSignal West Pico PLUS ECL kit (Thermo-Fisher) and visualized using the Azure Biosystems c280 imager. Band density was quantified using ImageJ version 1.53.

### Statistical Analyses

For graphs, data are shown as mean±SEM, and unless otherwise indicated, statistical differences were evaluated using a Student's t test with Welch's correction to assume a non-normal variance in our data distribution. Samples were compared using the Log-rank (Mantel-Cox) test with correction for testing multiple hypotheses. p <0.05 was considered significant. Graphs were produced and statistical analyses were performed using GraphPad Prism.

### REFERENCES

1. Henning, A.N., Roychoudhuri, R. & Restifo, N.P. Epigenetic control of CD8 (+) T cell differentiation. Nat Rev Immunol 18, 340-356 (2018).
2. Youngblood, B., Hale, J.S. & Ahmed, R. T-cell memory differentiation: insights from transcriptional signatures and epigenetics. Immunology 139, 277-284 (2013).
3. Bantug, G.R., Galluzzi, L., Kroemer, G. & Hess, C. The spectrum of T cell metabolism in health and disease. Nat Rev Immunol 18, 19-34 (2018).
4. Chang, J.T., Wherry, E.J. & Goldrath, A. W. Molecular regulation of effector and memory T cell differentiation. Nat Immunol 15, 1104-1115 (2014).
5. Kaech, S.M. & Cui, W. Transcriptional control of effector and memory CD8+ T cell differentiation. Nat Rev Immunol 12, 749-761 (2012).
6. Kaech, S.M. et al. Selective expression of the interleukin 7 receptor identifies effector CD8 T cells that give rise to long-lived memory cells. Nat Immunol 4, 1191-1198 (2003).
7. Joshi, N.S. et al. Inflammation directs memory precursor and short-lived effector CD8(+) T cell fates via the graded expression of T-bet transcription factor. Immunity 27, 281-295 (2007).
8. Sarkar, S. et al. Functional and genomic profiling of effector CD8 T cell subsets with distinct memory fates. J Exp Med 205, 625-640 (2008).
9 Biron, C.A., Byron, K.S. & Sullivan, J.L. Severe herpesvirus infections in an adolescent without natural killer cells. N Engl J Med 320, 1731-1735 (1989).
10. Bukowski, J.F., Warner, J.F., Dennert, G. & Welsh, R.M. Adoptive transfer studies demonstrating the antiviral effect of natural killer cells in vivo. J Exp Med 161, 40-52 (1985).
11. Weizman, O.E. et al. ILC1 Confer Early Host Protection at Initial Sites of Viral Infection. Cell 171, 795-808 e712 (2017).
12. Riggan, L., Freud, A.G. & O'Sullivan, T.E. True Detective: Unraveling Group 1 Innate Lymphocyte Heterogeneity. Trends Immunol 40, 909-921 (2019).
13. Weizman, O.E. et al. Mouse cytomegalovirus-experienced ILC1s acquire a memory response dependent on the viral glycoprotein m12. Nat Immunol 20, 1004-1011 (2019).
14. Sun, J.C., Beilke, J.N. & Lanier, L.L. Adaptive immune features of natural killer cells. Nature 457, 557-561 (2009).
15. O'Sullivan, T.E., Sun, J.C. & Lanier, L.L. Natural Killer Cell Memory. Immunity 43, 634-645 (2015).
16. Grassmann, S. et al. Distinct Surface Expression of Activating Receptor Ly49H Drives Differential Expansion of NK Cell Clones upon Murine Cytomegalovirus Infection. Immunity 50, 1391-1400 e1394 (2019).
17. Arase, H., Mocarski, E.S., Campbell, A.E., Hill, A.B. & Lanier, L.L. Direct recognition of cytomegalovirus by activating and inhibitory NK cell receptors. Science 296, 1323-1326 (2002).
18. Brown, M.G. et al. Vital involvement of a natural killer cell activation receptor in resistance to viral infection. Science 292, 934-937 (2001).
19. Crinier, A. et al. High-Dimensional Single-Cell Analysis Identifies Organ-Specific Signatures and Conserved NK Cell Subsets in Humans and Mice. Immunity 49, 971-986 e975 (2018).
20. Horowitz, A. et al. Genetic and environmental determinants of human NK cell diversity revealed by mass cytometry. Sci Transl Med 5, 208ra145 (2013).
21. Karo, J.M., Schatz, D.G. & Sun, J.C. The RAG recombinase dictates functional heterogeneity and cellular fitness in natural killer cells. Cell 159, 94-107 (2014).
22. Rahim, M.M. et al. Expansion and Protection by a Virus-Specific NK Cell Subset Lacking Expression of the Inhibitory NKR-PIB Receptor during Murine Cytomegalovirus Infection. J Immunol 197, 2325-2337 (2016).
23. Kamimura, Y. & Lanier, L.L. Homeostatic control of memory cell progenitors in the natural killer cell lineage. Cell Rep 10, 280-291 (2015).
24. Adams, N.M., Diaz-Salazar, C., Dang, C., Lanier, L.L. & Sun, J.C. Cutting Edge: Heterogeneity in Cell Age Contributes to Functional Diversity of NK Cells. J Immunol 206, 465-470 (2021).
25. Min-Oo, G., Bezman, N.A., Madera, S., Sun, J.C. & Lanier, L.L. Proapoptotic Bim regulates antigen-specific NK cell contraction and the generation of the memory NK cell pool after cytomegalovirus infection. J Exp Med 211, 1289-1296 (2014).
26. La Manno, G. et al. RNA velocity of single cells. Nature 560, 494-498 (2018).
27. van Helden, M.J. et al. Terminal NK cell maturation is controlled by concerted actions of T-bet and Zeb2 and is essential for melanoma rejection. J Exp Med 212, 2015-2025 (2015).
28. Arguello, R.J. et al. SCENITH: A Flow Cytometry-Based Method to Functionally Profile Energy Metabolism with Single-Cell Resolution. Cell Metab 32, 1063-1075 e1067 (2020).
29. O'Sullivan, T.E., Johnson, L.R., Kang, H.H. & Sun, J.C. BNIP3-and BNIP3L-Mediated Mitophagy Promotes the Generation of Natural Killer Cell Memory. Immunity 43, 331-342 (2015).
30. Bezman, N.A. et al. Molecular definition of the identity and activation of natural killer cells. Nat Immunol 13, 1000-1009 (2012).
31. Min-Oo, G. & Lanier, L.L. Cytomegalovirus generates long-lived antigen-specific NK cells with diminished bystander activation to heterologous infection. J Exp Med 211, 2669-2680 (2014).
32. Johnnidis, J.B. et al. Inhibitory signaling sustains a distinct early memory CD8(+) T cell precursor that is resistant to DNA damage. Sci Immunol 6 (2021).
33. Viant, C. et al. Cell cycle progression dictates the requirement for BCL2 in natural killer cell survival. J Exp Med 214, 491-510 (2017).
34. Riggan, L. et al. CRISPR-Cas9 Ribonucleoprotein-Mediated Genomic Editing in Mature Primary Innate Immune Cells. Cell Rep 31, 107651 (2020).
35. Wiedemann, G.M. et al. Deconvoluting global cytokine signaling networks in natural killer cells. Nat Immunol 22, 627-638 (2021).
36. Kurtulus, S. et al. Bcl-2 allows effector and memory CD8+ T cells to tolerate higher expression of Bim. J Immunol 186, 5729-5737 (2011).
37. Dunkle, A., Dzhagalov, I., Gordy, C. & He, Y.W. Transfer of CD8+ T cell memory using Bcl-2 as a marker. J Immunol 190, 940-947 (2013).
38. Sathe, P. et al. Innate immunodeficiency following genetic ablation of Mcl1 in natural killer cells. Nat Commun 5, 4539 (2014).
39. Mujal, A.M., Delconte, R.B. & Sun, J.C. Natural Killer Cells: From Innate to Adaptive Features. Annu Rev Immunol 39, 417-447 (2021).
40. Omilusik, K.D. et al. Transcriptional repressor ZEB2 promotes terminal differentiation of CD8+ effector and memory T cell populations during infection. J Exp Med 212, 2027-2039 (2015).
41. Dominguez, C.X. et al. The transcription factors ZEB2 and T-bet cooperate to program cytotoxic T cell terminal differentiation in response to LCMV viral infection. J Exp Med 212, 2041-2056 (2015).
42. Omilusik, K.D. et al. Sustained Id2 regulation of E proteins is required for terminal differentiation of effector CD8(+) T cells. J Exp Med 215, 773-783 (2018).
43. Delconte, R.B. et al. The Helix-Loop-Helix Protein ID2 Governs NK Cell Fate by Tuning Their Sensitivity to Interleukin-15. Immunity 44, 103-115 (2016).
44. Ikawa, T., Fujimoto, S., Kawamoto, H., Katsura, Y. & Yokota, Y. Commitment to natural killer cells requires the helix-loop-helix inhibitor Id2. Proc Natl Acad Sci U S A 98, 5164-5169 (2001).
45. Zook, E.C. et al. Transcription factor ID2 prevents E proteins from enforcing a naive T lymphocyte gene program during NK cell development. Sci Immunol 3 (2018).
46. Madera, S. et al. Cutting Edge: Divergent Requirement of T-Box Transcription Factors in Effector and Memory NK Cells. J Immunol 200, 1977-1981 (2018).
47. Rapp, M. et al. Core-binding factor beta and Runx transcription factors promote adaptive natural killer cell responses. Sci Immunol 2 (2017).
48. Taveirne, S. et al. The transcription factor ETS1 is an important regulator of human NK cell development and terminal differentiation. Blood 136, 288-298 (2020).
49. Grund, E.M., Spyropoulos, D.D., Watson, D.K. & Muise-Helmericks, R.C. Interleukins 2 and 15 regulate Ets1 expression via ERK1/2 and MNK1 in human natural killer cells. J Biol Chem 280, 4772-4778 (2005).
50. Ramirez, K. et al. Gene deregulation and chronic activation in natural killer cells deficient in the transcription factor ETS1. Immunity 36, 921-932 (2012).
51. Chen, Z. et al. In vivo CD8(+) T cell CRISPR screening reveals control by Fli1 in infection and cancer. Cell 184, 1262-1280 e1222 (2021).
52. Wang, T. et al. Gene Essentiality Profiling Reveals Gene Networks and Synthetic Lethal Interactions with Oncogenic Ras. Cell 168, 890-903 e815 (2017).
53. Shen, M.W. et al. Predictable and precise template-free CRISPR editing of pathogenic variants. Nature 563, 646-651 (2018).
54. Hildreth, A.D., Riggan, L. & O'Sullivan, T.E. CRISPR-Cas9 Ribonucleoprotein-Mediated Genomic Editing in Primary Innate Immune Cells. STAR Protoc 1, 100113 (2020).
55. Deng, J. et al. BH3 profiling identifies three distinct classes of apoptotic blocks to predict response to ABT-737 and conventional chemotherapeutic agents. Cancer Cell 12, 171-185 (2007).
56. Stuart, T. et al. Comprehensive Integration of Single-Cell Data. Cell 177, 1888-1902 e1821 (2019).
57. Bergen, V., Lange, M., Peidli, S., Wolf, F.A. & Theis, F.J. Generalizing RNA velocity to transient cell states through dynamical modeling. Nat Biotechnol 38, 1408-1414 (2020).
58. Trapnell, C. et al. The dynamics and regulators of cell fate decisions are revealed by pseudotemporal ordering of single cells. Nat Biotechnol 32, 381-386 (2014).
59. Dobin, A. et al. STAR: ultrafast universal RNA-seq aligner. Bioinformatics 29, 15-21 (2013).
60. Love, M.I., Huber, W. & Anders, S. Moderated estimation of fold change and dispersion for RNA-seq data with DESeq2. Genome Biol 15, 550 (2014).
61. Yu, B. et al. Epigenetic landscapes reveal transcription factors that regulate CD8(+) T cell differentiation. Nat Immunol 18, 573-582 (2017).
62. Kechin, A., Boyarskikh, U., Kel, A. & Filipenko, M. cutPrimers: A New Tool for Accurate Cutting of Primers from Reads of Targeted Next Generation Sequencing. J Comput Biol 24, 1138-1143 (2017).
63. Langmead, B. & Salzberg, S.L. Fast gapped-read alignment with Bowtie 2. Nat Methods 9, 357-359 (2012).
64. Zhang, Y. et al. Model-based analysis of ChIP-Seq (MACS). Genome Biol 9, R137 (2008).
65. Anders, S., Pyl, P.T. & Huber, W. HTSeq—a Python framework to work with high-throughput sequencing data. Bioinformatics 31, 166-169 (2015).
66. Heinz, S. et al. Simple combinations of lineage-determining transcription factors prime cis-regulatory elements required for macrophage and B cell identities. Mol Cell 38, 576-589 (2010).

Additional electroporation methods, materials and devices that can be used with embodiments of the invention are disclosed, for example in US Patent Application Publication Nos.: 20200362355, 20200048606, 20200000851 and 20190388469, as well as literature references such as: Functional CRISPR dissection of gene networks controlling human regulatory T cell identity. Schumann et al. Nat Immunol (2020); CRISPR screen in regulatory T cells reveals modulators of Foxp3; Cortez et al., Nature 2020, 29 Apr.; Pooled Knockin Targeting for Genome Engineering of Cellular

Immunotherapies. Roth et al., Cell 2020 Apr. 30; Polymer-stabilized Cas9 nanoparticles and modified repair templates increase genome editing efficiency. Nguyen et al., Nat Biotechnol. 2019 Dec. 9; Landscape of stimulation-responsive chromatin across diverse human immune cells; Calderon et al., Nat Genet. 2019 Sep. 30; Large dataset enables prediction of repair after CRISPR-Cas9 editing in primary T cells; Leenay et al., Nat Biotechnol. 2019 September;37(9):1034-1037; A large CRISPR-induced bystander mutation causes immune dysregulation; Simeonov et al., Commun Biol. 2019 Feb. 18;2:70; Genome-wide CRISPR Screens in Primary Human T Cells Reveal Key Regulators of Immune Function. Shifrut et al., Cell 2018 December; 175:1-14; Reprogramming human T cell function and specificity with non-viral genome targeting; Roth et al., Nature. 2018 July;559(7714):405-409; “T-bet”-ing on autoimmunity variants. Nguyen et al., PLOS Genetics. 13(9); e1006924 (2017); Discovery of stimulation-responsive immune enhancers with CRISPR activation; Simeonov et al., Nature. 549; 111-115 (2017); A Cas9 Ribonucleoprotein Platform for Functional Genetic Studies of HIV-Host Interactions in Primary Human Cells. Hulquist et al., Cell Reports. 17; 138-1452 (2016); and Generation of Knock-in Primary Human T Cells Using Cas9 Ribonucleoproteins Schumamn et al., PNAS. (2015), the contents of all of which are incorporated herein by reference.

All publications mentioned herein such as Riggan et al., Nature Immunology volume 23, pages 556-567 (2022) (this publication is also referred to herein as “Riggan et al.”); PCT Publication Serial No. PCT/US21/62365, and those identified above are incorporated by reference to disclose and describe aspects, methods and/or materials in connection with the cited publications.

Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of decreasing levels of Bcl-2-like protein 11 (“BIM”) observed in early effector NK cells following viral infection, the method comprising perturbing a Friend leukemia integration 1 transcription factor (“FLI1”) gene in the early effector NK cells such that levels of BIM in the early effector NK cells are observed to be decreased following viral infection as compared to early effector NK cells not having a perturbation in the FLI1 gene.

2. The method of claim 1, wherein perturbing the FLI1 gene comprises making a deletion in the FLI1 gene made using a clustered regularly interspaced short palindromic repeats (CRISPR) process comprising:
(a) combining a CRISPR ribonucleoprotein complex with the early effector NK cells;
(b) electroporating the combination of (a) under conditions comprising:
a voltage between 1700-2000 volts; and
a pulse width of width of about 1×20-30 milliseconds;
such that the CRISPR ribonucleoprotein complex is electroporated into the early effector NK cells.

3. The method of claim 1, wherein the early effector NK cell is selected to be a mature naive NK cell.

4. The method of claim 1, wherein the early effector NK cell is disposed within a culture media comprising one or more cytokines, wherein the one or more cytokines is selected from: IL-2, IL-3, IL-4, IL-15, the notch ligand DLL1, stem cell factor (SCF), FLT3 ligand (FLT3L), thrombopoietin (TPO), GM-CSF, and M-CSF.

5. The method of claim 1, wherein the early effector NK cell comprises a further modification to genomic DNA comprising a further deletion of one or more genes in the early effector NK cell and/or the addition of one or more genes in the early effector NK cell.

6. An isolated NK cell having a deletion in a Friend leukemia integration 1 transcription factor (“FLI1”) gene such that levels of Bcl-2-like protein 11 (“BIM”) in the NK cell are decreased.

7. The NK cell of claim 6, wherein the NK cell is a mature naive NK cell.

8. The NK cell of claim 6, wherein the cell is disposed within a culture media comprising one or more cytokines, wherein the one or more cytokines is selected from: IL-2, IL-3, IL-4, IL-15, the notch ligand DLL1, stem cell factor (SCF), FLT3 ligand (FLT3L), thrombopoietin (TPO), GM-CSF, and M-CSF.

9. The NK cell of claim 6, wherein the cell comprises a further modification to genomic DNA comprising a further deletion of one or more genes in the NK cell and/or the addition of one or more genes in the NK cell.

10. A method of making a genetically modified NK cell, the methods comprising making a perturbation in a Friend leukemia integration 1 transcription factor (“FLI1”) gene of an NK cell so that the genetically modified NK cell is made.

11. The method of claim 10, wherein the perturbation in the FLI1 gene is made using a clustered regularly interspaced short palindromic repeats (CRISPR) process.

12. The method of claim 11, wherein the CRISPR process comprises:
(a) combining a CRISPR ribonucleoprotein complex with the NK cells;
(b) electroporating the combination of (a) under conditions comprising:
a voltage between 1700-2000 volts; and
a pulse width of width of about 1×20-30 milliseconds;
such that the CRISPR ribonucleoprotein complex is electroporated into the NK cells.

13. The method of claim 10, wherein the NK cells are combined with one or more cytokines following collection and prior to electroporation; wherein the one or more cytokines is selected from: IL-2, IL-3, IL-4, IL-15, the notch ligand DLL1, stem cell factor (SCF), FLT3 ligand (FLT3L), thrombopoietin (TPO), GM-CSF, and M-CSF.

14. The method of claim 10, wherein the method comprises deleting the FLI1 gene in the early effector NK cells such that levels of the pro-apoptotic factor Bim in the early effector NK cells are decreased.

15. The method of claim 10, wherein the NK cell is a primary cell obtained from the peripheral blood leukocytes of an individual.
